(19) United States
(12) Patent Application Publication
(10) Pub. No.: US 2011/0077628 A1
(43) Pub. Date: Mar. 31, 2011

(54) MEDICAL SYSTEM AND METHOD OF USE

(75) Inventors: Michael HOEY, Shoreview, MN (US); John H. SHADDUCK, Berkeley, CA (US)

(73) Assignee: Tsunami MedTech, LLC, Menlo Park, CA (US)

(21) Appl. No.: 12/856,339

(22) Filed: Aug. 13, 2010

Related U.S. Application Data
(63) Continuation-in-part of application No. 11/329,381, filed on Jan. 10, 2006.
(60) Provisional application No. 61/274,162, filed on Aug. 13, 2009.

(51) Int. Cl. A61B 18/04; A61F 7/12

(52) U.S. Cl. 606/27; 607/105

(57) ABSTRACT

An instrument and method for tissue thermotherapy including an inductive heating means to generate a vapor phase media that is used for interstitial, intraluminal, intracavity or topical tissue treatment. In one method, the vapor phase media is propagated from a probe outlet to provide a controlled vapor-to-liquid phase change in an interface with tissue to thereby apply ablative thermal energy delivery.

### CROSS-REFERENCE TO RELATED APPLICATIONS

This application is a non-provisional of U.S. Patent Application No. 61/274,162 filed on Aug. 13, 2009, the content of which is incorporated herein by reference in its entirety

### FIELD OF THE INVENTION

This invention relates to medical instruments and systems for applying energy to tissue, and more particularly relates to a system for ablating, sealing, welding, coagulating, shrinking or creating lesions in tissue by means of contacting a targeted in a patient with a vapor phase media wherein a subsequent vapor-to-liquid phase change of the media applies thermal energy to the tissue to cause an intended therapeutic effect. Variations of the invention include devices and methods for generating a flow of high quality vapor and monitoring the vapor flow for various parameters with one or more sensors. In yet additional variations, the invention includes devices and methods for modulating parameters of the system in response to the observed parameters.

### BACKGROUND OF THE INVENTION

Various types of medical instruments utilizing radiofrequency (Rf) energy, laser energy, microwave energy and the like have been developed for delivering thermal energy to tissue, for example to ablate tissue. While such prior art forms of energy delivery work well for some applications, Rf, laser and microwave energy typically cannot cause highly “controlled” and “localized” thermal effects that are desirable in controlled ablation soft tissue for ablating a controlled depth or for the creation of precise lesions in such tissue. In general, the non-linear or non-uniform characteristics of tissue affect electromagnetic energy distributions in tissue.

What is needed are systems and methods that controllably apply thermal energy in a controlled and localized manner without the lack of control often associated when Rf, laser and microwave energy are applied directly to tissue.

This application is related to the following U.S. Non-provisional and Provisional applications: Application No. 61/126,647 Filed on May 6, 2008 MEDICAL SYSTEM AND METHOD OF USE (docket TSMT-P-T004.20-US); Application No. 61/126,651 Filed on May 6, 2008 MEDICAL SYSTEM AND METHOD OF USE (docket TSMT-P-T004.40-US); TSMT-P-T004.50-U.S. Application No. 61/126,612 Filed on May 6, 2008 MEDICAL SYSTEM AND METHOD OF USE (docket TSMT-P-T004.40-US); Application No. 61/126,636 Filed on May 6, 2008 MEDICAL SYSTEM AND METHOD OF USE (docket TSMT-P-T004.60-US; Application No. 61/130,345 Filed on May 31, 2008 MEDICAL SYSTEM AND METHOD OF USE (docket TSMT-P-T004.70-US); Application No. 61/191,459 Filed on Sep. 9, 2008 MEDICAL SYSTEM AND METHOD OF USE (docket TSMT-P-T005.50-US); Application No. 61/066,396 Filed on Feb. 20, 2008 TISSUE ABLATION SYSTEM AND METHOD OF USE (TSMT-P-T005.60-US); Application No. 61/123,416 Filed on Apr. 8, 2008 MEDICAL SYSTEM AND METHOD OF USE (TSMT-P-T005.70-US); Application No. 61/068,049 Filed on Mar. 4, 2008 MEDICAL SYSTEM AND METHOD OF USE (TSMT-P-T005.80-US); Application No. 61/123,384 Filed on Apr. 8, 2008 MEDICAL SYSTEM AND METHOD OF USE (TSMT-P-T005.90-US); Application No. 61/068,130 Filed on Mar. 4, 2008 MEDICAL SYSTEM AND METHOD OF USE (docket TSMT-P-T006.00-US); Application No. 61/123,417 Filed on Apr. 8, 2008 MEDICAL SYSTEM AND METHOD OF USE (docket TSMT-P-T006.10-US); Application No. 61/123,412 Filed on Apr. 8, 2008 MEDICAL SYSTEM AND METHOD OF USE (docket TSMT-P-T006.20-US); Application No. 61/126,830 Filed on May 7, 2008 MEDICAL SYSTEM AND METHOD OF USE (docket TSMT-P-T006.40-US); and Application No. 61/126,620 Filed on May 6, 2008 MEDICAL SYSTEM AND METHOD OF USE (docket TSMT-P-T006.50-US).

The systems and methods described herein are also related to U.S. patent application Ser. No. 10/681,625 filed Oct. 7, 2003 titled “Medical Instruments and Techniques for Thermally-Mediated Therapies”; Ser. No. 11/158,930 filed Jun. 22, 2005 titled “Medical Instruments and Techniques for Treating Pulmonary Disorders”; Ser. No. 11/244,329 (Docket No. S-TT-00200A) filed Oct. 5, 2005 titled “Medical Instruments and Methods of Use” and Ser. No. 11/329,381 (Docket No. S-TT-00300A) filed Jan. 10, 2006 titled “Medical Instrument and Method of Use”.

All of the above applications are incorporated herein by this reference and made a part of this specification, together with the specifications of all other commonly-invented applications cited in the above applications.

### SUMMARY OF THE INVENTION

The present invention is adapted to provide improved methods of controlled thermal energy delivery to localized tissue volumes, for example for ablating, sealing, coagulating or otherwise damaging targeted tissue, for example to ablate a tissue volume interstitially or to ablate the lining of a body cavity. Of particular interest, the method causes thermal effects in targeted tissue without the use of Rf current flow through the patient's body and without the potential of carbonizing tissue.

In general, the thermally-mediated treatment method comprises causing a vapor-to-liquid phase state change in a selected media at a targeted tissue site thereby applying thermal energy substantially equal to the heat of vaporization of the selected media to the tissue site. The thermally-mediated therapy can be delivered to tissue by such vapor-to-liquid phase transitions, or “internal energy” releases, about the working surfaces of several types of instruments for ablative treatments of soft tissue. FIGS. 1A and 1B illustrate the phenomena of phase transitional releases of internal energies. Such internal energy involves energy on the molecular and atomic scale—and in polyatomic gases is directly related to intermolecular attractive forces, as well as rotational and vibrational kinetic energy. In other words, the method of the invention exploits the phenomenon of internal energy transitions between gaseous and liquid phases that involve very large amounts of energy compared to specific heat.

It has been found that the controlled application of such energy in a controlled media-tissue interaction solves many of the vexing problems associated with energy-tissue interactions in Rf, laser and ultrasound modalities. The apparatus of the invention provides a vaporization chamber in the interior of an instrument, in an instrument working end or in a source remote from the instrument end. A source provides liquid media to the interior vaporization chamber wherein energy is applied to create a selected volume of vapor media. In the process of the liquid-to-vapor phase transition of a liquid media, for example water, large amounts of energy are added to overcome the cohesive forces between molecules in the liquid, and an additional amount of energy is required to expand the liquid 1000+ percent (PAD) into a resulting vapor phase (see FIG. 1A). Conversely, in the vapor-to-liquid transition, such energy will be released at the phase transition at the interface with the targeted tissue site. That is, the heat of vaporization is released at the interface when the media transitions from gaseous phase to liquid phase wherein the random, disordered motion of molecules in the vapor regain cohesion to convert to a liquid media. This release of energy (defined as the capacity for doing work) relating to intermolecular attractive forces is transformed into therapeutic heat for a thermotherapy at the interface with the targeted body structure. Heat flow and work are both ways of transferring energy.

In FIG. 1A, the simplified visualization of internal energy is useful for understanding phase transition phenomena that involve internal energy transitions between liquid and vapor phases. If heat were added at a constant rate in FIG. 1A (graphically represented as 5 calories/gm blocks) to elevate the temperature of water through its phase change to a vapor phase, the additional energy required to achieve the phase change (latent heat of vaporization) is represented by the large number of 110+ blocks of energy at 100° C. in FIG. 1A. Still referring to FIG. 1A, it can be easily understood that all other prior art ablation modalities—Rf, laser, microwave and ultrasound—create energy densities by simply ramping up calories/gm as indicated by the temperature range from 37° C. through 100° C. as in FIG. 1A. The prior art modalities make no use of the phenomenon of phase transition energies as depicted in FIG. 1A.

FIG. 1B graphically represents a block diagram relating to energy delivery aspects of the present invention. The system provides for insulative containment of an initial primary energy-media interaction within an interior vaporization chamber of medical thermotherapy system. The initial, ascendant energy-media interaction delivers energy sufficient to achieve the heat of vaporization of a selected liquid media, such as water or saline solution, within an interior of the system. This aspect of the technology requires a highly controlled energy source wherein a computer controller may need to modulated energy application between very large energy densities to initially surpass the latent heat of vaporization with some energy sources (e.g. a resistive heat source, an Rf energy source, a light energy source, a microwave energy source, an ultrasound source and/or an inductive heat source) and potential subsequent lesser energy densities for maintaining a high vapor quality. Additionally, a controller must control the pressure of liquid flows for replenishing the selected liquid media at the required rate and optionally for controlling propagation velocity of the vapor phase media from the working end surface of the instrument. In use, the method of the invention comprises the controlled application of energy to achieve the heat of vaporization as in FIG. 1A and the controlled vapor-to-liquid phase transition and vapor exit pressure to thereby control the interaction of a selected volume of vapor at the interface with tissue. The vapor-to-liquid phase transition can deposit 400, 500, 600 or more cal/gram within the targeted tissue site to perform the thermal ablation with the vapor in typical pressures and temperatures.

In one variation, the present disclosure includes medical systems for applying thermal energy to tissue, where the system comprises an elongated probe with an axis having an interior flow channel extending to at least one outlet in a probe working end; a source of vapor media configured to provide a vapor flow through at least a portion of the interior flow channel, wherein the vapor has a minimum temperature; and at least one sensor in the flow channel for providing a signal of at least one flow parameter selected from the group one of (i) existence of a flow of the vapor media, (ii) quantification of a flow rate of the vapor media, and (iii) quality of the flow of the vapor media. The medical system can include variations where the minimum temperature varies from at least 80° C., 100° C. 120° C., 140° C. and 160° C. However, other temperature ranges can be included depending upon the desired application.

Sensors included in the above system include temperature sensor, an impedance sensor, a pressure sensor as well as an optical sensor.

The source of vapor media can include a pressurized source of a liquid media and an energy source for phase conversion of the liquid media to a vapor media. In addition, the medical system can further include a controller capable of modulating a vapor parameter in response to a signal of a flow parameter; the vapor parameter selected from the group of (i) flow rate of pressurized source of liquid media, (ii) inflow pressure of the pressurized source of liquid media, (iii) temperature of the liquid media, (iv) energy applied from the energy source to the liquid media, (v) flow rate of vapor media in the flow channel, (vi) pressure of the vapor media in the flow channel, (vi) temperature of the vapor media, and (vii) quality of vapor media.

In another variation, a novel medical system for applying thermal energy to tissue comprises an elongated probe with an axis having an interior flow channel extending to at least one outlet in a probe working end, wherein a wall of the flow channel includes an insulative portion having a thermal conductivity of less than a maximum thermal conductivity; and a source of vapor media configured to provide a vapor flow through at least a portion of the interior flow channel, wherein the vapor has a minimum temperature.

Variations of such systems include systems where the maximum thermal conductivity ranges from 0.05 W/mK, 0.01 W/mK and 0.005 W/mK.

Methods are disclosed herein for thermally treating tissue by providing a probe body having a flow channel extending therein to an outlet in a working end, introducing a flow of a liquid media through the flow channel and applying energy to the tissue by inductively heating a portion of the probe sufficient to vaporize the flowing media within the flow channel causing pressurized ejection of the media from the outlet to the tissue.

The methods can include applying energy between 10 and 10,000 Joules to the tissue from the media. The rate at which the media flows can be controlled as well. In

The method of claim 1 where introducing the flow of liquid media comprises introducing the flow of liquid media in less than 10 minutes. However, the rate can be reduced as described below.

In another variation, the methods described herein include inductively heating the portion of the probe by applying an electromagnetic energy source to a coil surrounding the flow channel. The electromagnetic energy can also inductively heat a wall portion of the flow channel.

Another variation of the method includes providing a flow permeable structure within the flow channel. Optionally, the coil described herein can heat the flow permeable structure to transfer energy to the flow media. Some examples of a flow permeable structure include woven filaments, braided filaments, knit filaments, metal wool, a microchannel structure, a porous structure, a honeycomb structure and an open cell structure. However, any structure that is permeable to flow can be included.

The electromagnetic energy source can include an energy source ranging from a 10 Watt source to a 500 Watt source.

Medical systems for treating tissue are also described herein. Such systems can include a probe body having a flow channel extending therein to an outlet in a working end, a coil about at least a portion or the flow channel, and an electromagnetic energy source coupled to the coil, where the electromagnetic energy source induces current in the coil causing energy delivery to a flowable media in the flow channel. The systems can include a source of flowable media coupled to the flow channel. The electromagnetic energy source can be capable of applying energy to the flowable media sufficient to cause a liquid-to-vapor phase change in at least a portion of the flowable media as described in detail herein. In addition the probe can include a sensor selected from a temperature sensor, an impedance sensor, a capacitance sensor and a pressure sensor. In some variations the probe is coupled to an aspiration source.

The medical system can also include a controller capable of modulating at least one operational parameter of the source of flowable media in response to a signal from a sensor. For example, the controller can be capable of modulating a flow of the flowable media. In another variation, the controller is capable of modulating a flow of the flowable media to apply between 100 and 10,000 Joules to the tissue.

The systems described herein can also include a metal portion in the flow channel for contacting the flowable media. The metal portion can be a flow permeable structure and can optionally comprise a microchannel structure. In additional variations, the flow permeable structure can include woven filaments, braided filaments, knit filaments, metal wool, a porous structure, a honeycomb structure, an open cell structure or a combination thereof.

In another variation, the methods described herein can include positioning a probe in an interface with a targeted tissue, and causing a vapor media from to be ejected from the probe into the interface with tissue wherein the media delivers energy ranging from 5 joules to 100,000 joules to cause a therapeutic effect, wherein the vapor media is converted from a liquid media within the probe by inductive heating means.

Methods described herein also include methods of treating tissue by providing medical system including a heat applicator portion for positioning in an interface with targeted tissue, and converting a liquid media into a vapor media within an elongated portion of the medical system having a flow channel communicating with a flow outlet in the heat applicator portion, and contacting the vapor media with the targeted tissue to thereby deliver energy ranging from 5 joules to 100,000 joules to cause a therapeutic effect.

As discussed herein, the methods can include converting the liquid into a vapor media using an inductive heating means. In an alternate variation, a resistive heating means can be combined with the inductive heating means or can replace the inductive heating means.

The instrument and method of the invention can cause an energy-tissue interaction that is imageable with intra-operative ultrasound or MRI.

The instrument and method of the invention cause thermal effects in tissue that do not rely applying an electrical field across the tissue to be treated.

Additional advantages of the invention will be apparent from the following description, the accompanying drawings and the appended claims.

All patents, patent applications and publications mentioned in this specification are herein incorporated by reference to the same extent as if each individual publication or patent application was specifically and individually indicated to be incorporated by reference.

In addition, it is intended that combinations of aspects of the systems and methods described herein as well as the various embodiments themselves, where possible, are within the scope of this disclosure.

### BRIEF DESCRIPTION OF THE DRAWINGS

FIG. 1A is a graphical depiction of the quantity of energy needed to achieve the heat of vaporization of water.

FIG. 1B is a diagram of phase change energy release that underlies a system and method of the invention.

FIG. 3 is a block diagram of a control method of the invention.

FIG. 4A is an illustration of the working end of FIG. 2 being introduced into soft tissue to treat a targeted tissue volume.

FIG. 4B is an illustration of the working end of FIG. 4A showing the propagation of vapor media in tissue in a method of use in ablating a tumor.

FIG. 5 is an illustration of a working end similar to FIGS. 4A-4B with vapor outlets comprising microporosities in a porous wall.

FIG. 6A is schematic view of a needle-type working end of a vapor delivery tool for applying energy to tissue.

FIG. 6B is schematic view of an alternative needle-type working end similar to FIG. 6A.

FIG. 6C is schematic view of a retractable needle-type working end similar to FIG. 6B.

FIG. 6D is schematic view of working end with multiple shape-memory needles.

FIG. 6E is schematic view of a working end with deflectable needles.

FIG. 6F is schematic view of a working end with a rotating element for directing vapor flows.

FIG. 6G is another view of the working end of FIG. 6F.

FIG. 6H is schematic view of a working end with a balloon.

FIG. 6I is schematic view of an articulating working end.

FIG. 6J is schematic view of an alternative working end with RF electrodes.

FIG. 6K is schematic view of an alternative working end with a resistive heating element.

FIG. 6L is schematic view of a working end with a tissue-capturing loop.

FIG. 6M is schematic view of an alternative working end with jaws for capturing and delivering vapor to tissue.

FIG. 7 is schematic view of an alternative working end with jaws for capturing and delivering vapor to tissue.

FIG. 8 is schematic view of an alternative working end with jaws for capturing and delivering vapor to tissue.

FIG. 9 is an partly disassembled view of a handle and inductive vapor generator system of the invention.

FIG. 10 is an enlarged schematic view of the inductive vapor generator of FIG. 9.

FIG. 11 is an illustration of a method of using a first embodiment of vapor delivery tool for treating an intervertebral disc.

FIG. 12A is a sectional view of the disc of FIG. 11 and an initial step of the method of introducing the vapor delivery tool of FIG. 11 into the disc.

FIG. 12B is a sectional view of the disc as in FIG. 11 depicting the vapor delivery tool deployed and showing the step of delivering vapor into the disc tissue.

FIG. 13 is an enlarged perspective view of the working end of the vapor delivery tool of FIGS. 12A-12B showing the configuration of vapor outlets.

FIG. 14 is an illustration of a method of using another embodiment of vapor delivery tool for treating an intervertebral disc similar to that of FIGS. 12A-12B, which includes the step of applying aspiration forces to the interior of the disc.

FIG. 15 is an enlarged perspective view of the working end of the vapor delivery tool of FIG. 14 showing the configuration of an aspiration channel.

FIG. 16 is an enlarged sectional view of the working end of another vapor delivery tool showing the configuration of an air-gap and gas flow channel for thermal insulation.

FIG. 17A is an illustration of a method of using another embodiment of vapor delivery tool for treating an intervertebral disc similar to that of FIG. 14, which includes first and second penetrating members for introducing vapor and applying aspiration forces, respectively.

FIG. 17B is a sectional view of the method of FIG. 17A depicting the first and second penetrating members introducing vapor and applying aspiration forces, respectively.

FIG. 18A is a view of an alternative working end of a vapor delivery tool that carries an elastomeric sleeve for preventing retrograde vapor flows in tissue.

FIG. 18B is a view of the working end of FIG. 18A in operation depicting the flexing of the elastomeric sleeve to capture and prevent retrograde vapor flows.

the disc as in FIG. 11 depicting the vapor delivery tool deployed and showing the step of delivering vapor into the disc tissue.

FIG. 19 is a view of an alternative vapor delivery tool configured for hammering into bone, such as a vertebra, for use in treating and ablating a basivertebral nerve.

FIG. 20 depicts a method corresponding to the invention utilizing the vapor delivery tool of FIG. 19, wherein vapor is directed to a central region of the vertebral body to ablate the basivertebral nerve.

FIG. 21 is a view of an alternative vapor delivery tool similar to that of FIG. 19 configured for hammering into bone.

FIG. 22 is a view of another vapor delivery tool similar to that of FIGS. 19 and 21 configured for hammering into bone.

FIG. 23 is a sectional view of the shaft of a vapor delivery tool of FIGS. 19, 21 and 22 with an insulative air gap.

FIG. 24 is a view of an alternative deflectable vapor delivery tool hammered into a vertebral body and a method of delivering vapor to ablate the basivertebral nerve.

FIG. 25A is a disassembled view of components of the deflectable vapor delivery tool of FIG. 24 which includes concentric rotatable shape memory sleeves.

FIG. 25B is an assembled view of the components of FIG. 25A showing the deflectable vapor delivery tool in a straight configuration.

FIG. 26 is a view of another vapor delivery tool hammered into a vertebral body and a method of delivering vapor to ablate the basivertebral nerve.

FIG. 27 is a view of another vapor delivery tool and method that includes independently deployable temperature sensing elements.

### DETAILED DESCRIPTION OF THE INVENTION

As used in the specification, “a” or “an” means one or more. As used in the claim(s), when used in conjunction with the word “comprising”, the words “a” or “an” mean one or more. As used herein, “another” means as least a second or more. “Substantially” or “substantial” mean largely but not entirely. For example, substantially may mean about 10% to about 99.999, about 25% to about 99.999% or about 50% to about 99.999%.

Treatment Liquid Source, Energy Source, Controller

Referring to FIG. 2, a schematic view of medical system 100 of the present invention is shown that is adapted for treating a tissue target, wherein the treatment comprises an ablation or thermotherapy and the tissue target can comprise any mammalian soft tissue to be ablated, sealed, contracted, coagulated, damaged or treated to elicit an immune response. The system 100 include an instrument or probe body 102 with a proximal handle end 104 and an extension portion 105 having a distal or working end indicated at 110. In one embodiment depicted in FIG. 2, the handle end 104 and extension portion 105 generally extend about longitudinal axis 115. In the embodiment of FIG. 2, the extension portion 105 is a substantially rigid tubular member with at least one flow channel therein, but the scope of the invention encompasses extension portions 105 of any mean diameter and any axial length, rigid or flexible, suited for treating a particular tissue target. In one embodiment, a rigid extension portion 105 can comprise a 20 Ga. to 40 Ga. needle with a short length for thermal treatment of a patient's cornea or a somewhat longer length for treating a patient's retina. In another embodiment, an elongate extension portion 105 of a vapor delivery tool can comprise a single needle or a plurality of needles having suitable lengths for tumor or soft tissue ablation in a liver, breast, gall bladder, prostate, bone and the like. In another embodiment, an elongate extension portion 105 can comprise a flexible catheter for introduction through a body lumen to access at tissue target, with a diameter ranging from about 1 to 10 mm. In another embodiment, the extension portion 105 or working end 110 can be articulatable, deflectable or deformable. The probe handle end 104 can be configured as a hand-held member, or can be configured for coupling to a robotic surgical system. In another embodiment, the working end 110 carries an openable and closeable structure for capturing tissue between first and second tissue-engaging surfaces, which can comprise actuatable components such as one or more clamps, jaws, loops, snares and the like. The proximal handle end 104 of the probe can carry various actuator mechanisms known in the art for actuating components of the system 100, and/or one or more footswitches can be used for actuating components of the system.

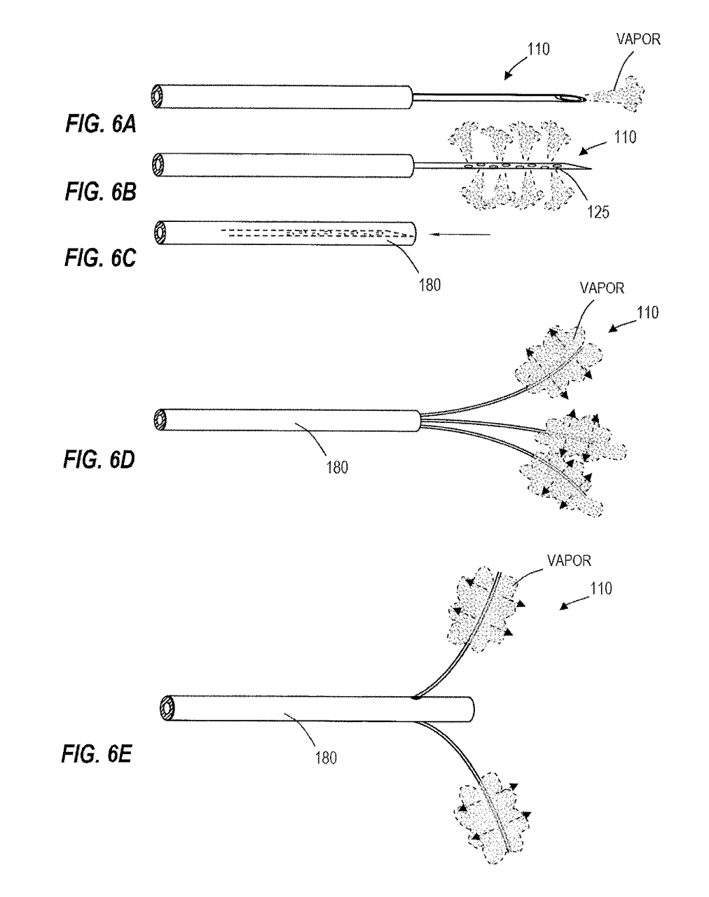

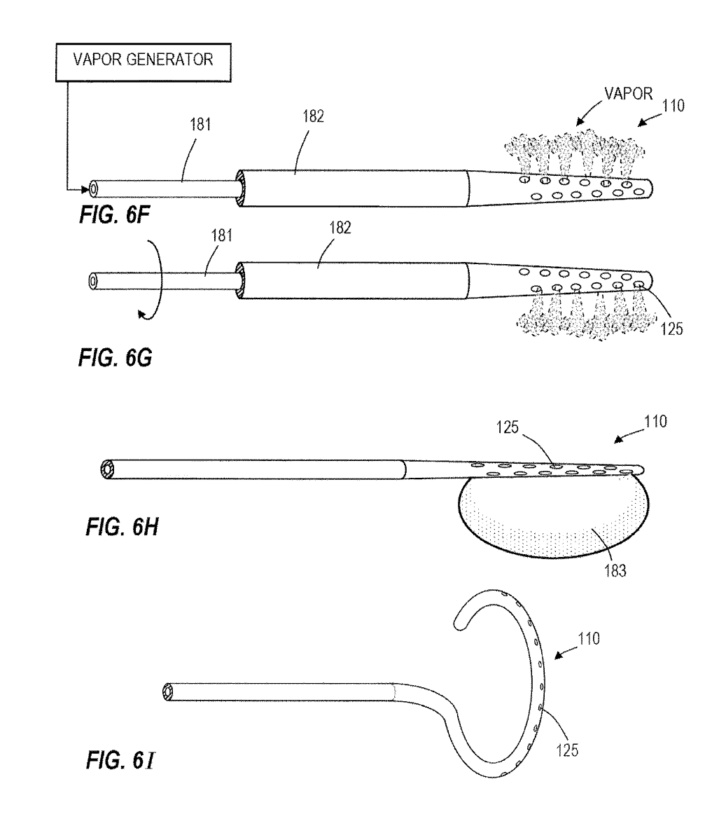

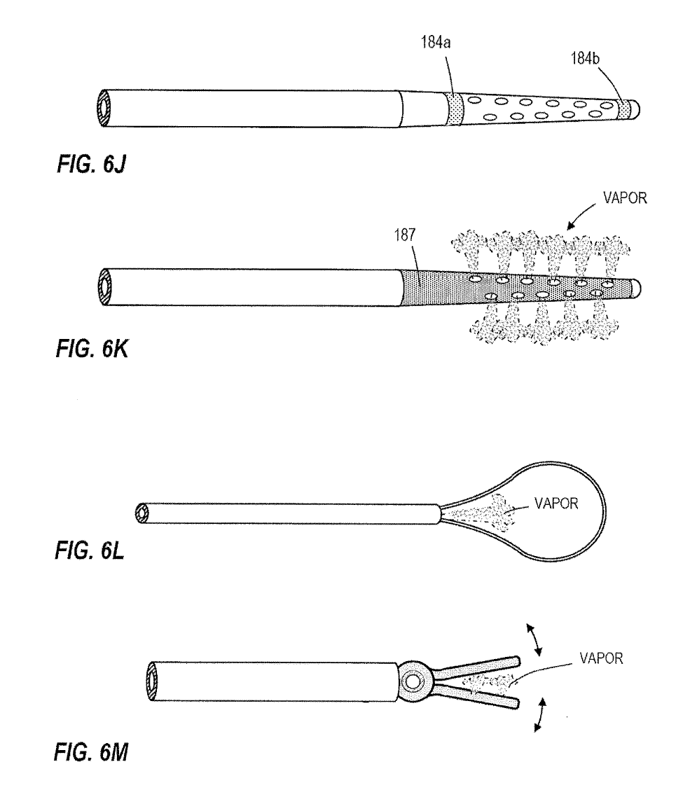

As can be seen in FIG. 2, the system 100 further includes a source 120 of a flowable liquid treatment media 121 that communicates with a flow channel 124 extending through the probe body 102 to at least one outlet 125 in the working end 110. The outlet 125 can be singular or multiple and have any suitable dimension and orientation as will be described further below. The distal tip 130 of the probe can be sharp for penetrating tissue, or can be blunt-tipped or open-ended with outlet 125. Alternatively, the working end 110 can be configured in any of the various embodiments shown in FIGS. 6A-6M and described further below.

In one embodiment shown in FIG. 2, an RF energy source 140 is operatively connected to a thermal energy source or emitter (e.g., opposing polarity electrodes 144a, 144b) in interior chamber 145 in the proximal handle end 104 of the probe for converting the liquid treatment media 121 from a liquid phase media to a non-liquid vapor phase media 122 with a heat of vaporization in the range of 60° C. to 200° C., or 80° C. to 120° C. A vaporization system using Rf energy and opposing polarity electrodes is disclosed in co-pending U.S. patent application Ser. No. 11/329,381 which is incorporated herein by reference. Another embodiment of vapor generation system is described in below in the Section titled “INDUCTIVE VAPOR GENERATION SYSTEMS”. In any system embodiment, for example in the system of FIG. 2, a controller 150 is provided that comprises a computer control system configured for controlling the operating parameters of inflows of liquid treatment media source 120 and energy applied to the liquid media by an energy source to cause the liquid-to-vapor conversion. The vapor generation systems described herein can consistently produce a high quality vapor having a temperature of at least 80° C., 100° C. 120° C., 140° C. and 160° C.

As can be seen in FIG. 2, the medical system 100 can further include a negative pressure or aspiration source indicated at 155 that is in fluid communication with a flow channel in probe 102 and working end 110 for aspirating treatment vapor media 122, body fluids, ablation by-products, tissue debris and the like from a targeted treatment site, as will be further described below. In FIG. 2, the controller 150 also is capable of modulating the operating parameters of the negative pressure source 155 to extract vapor media 122 from the treatment site or from the interior of the working end 110 by means of a recirculation channel to control flows of vapor media 122 as will be described further below.

In another embodiment, still referring to FIG. 2, medical system 100 further includes secondary media source 160 for providing an inflow of a second media, for example a biocompatible gas such as CO₂. In one method, a second media that includes at least one of depressurized CO₂, N₂, O₂ or H₂O can be introduced and combined with the vapor media 122. This second media 162 is introduced into the flow of non-ionized vapor media for lowering the mass average temperature of the combined flow for treating tissue. In another embodiment, the medical system 100 includes a source 170 of a therapeutic or pharmacological agent or a sealant composition indicated at 172 for providing an additional treatment effect in the target tissue. In FIG. 2, the controller indicated at 150 also is configured to modulate the operating parameters of source 160 and 170 to control inflows of a secondary vapor 162 and therapeutic agents, sealants or other compositions indicated at 172.

In FIG. 2, it is further illustrated that a sensor system 175 is carried within the probe 102 for monitoring a parameter of the vapor media 122 to thereby provide a feedback signal FS to the controller 150 by means of feedback circuitry to thereby allow the controller to modulate the output or operating parameters of treatment media source 120, energy source 140, negative pressure source 155, secondary media source 160 and therapeutic agent source 170. The sensor system 175 is further described below, and in one embodiment comprises a flow sensor to determine flows or the lack of a vapor flow. In another embodiment, the sensor system 175 includes a temperature sensor. In another embodiment, sensor system 175 includes a pressure sensor. In another embodiment, the sensor system 175 includes a sensor arrangement for determining the quality of the vapor media, e.g., in terms or vapor saturation or the like. The sensor systems will be described in more detail below.

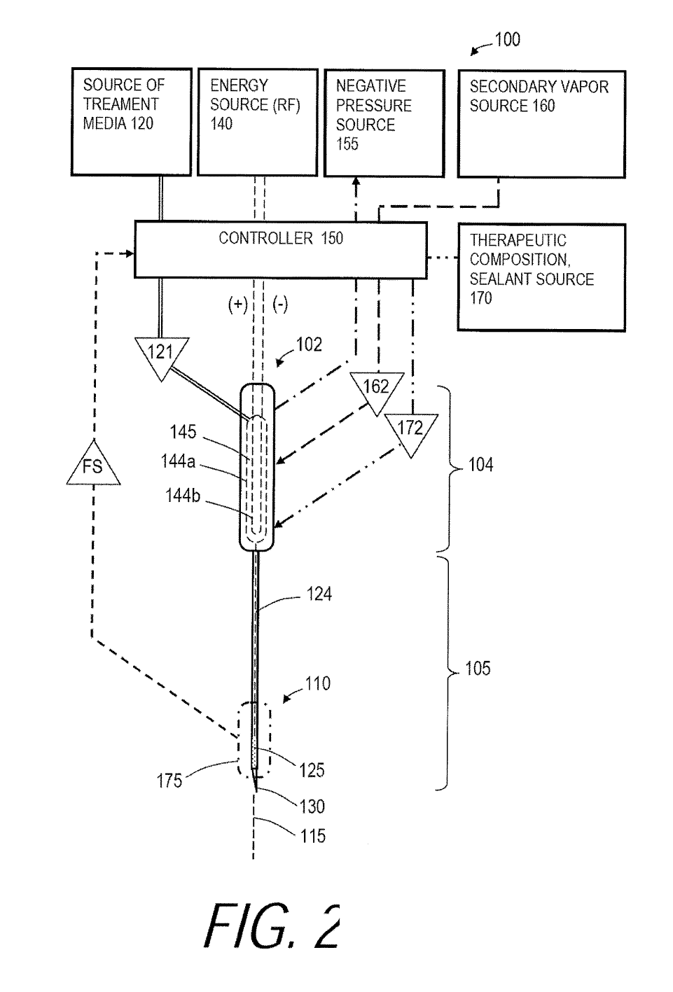

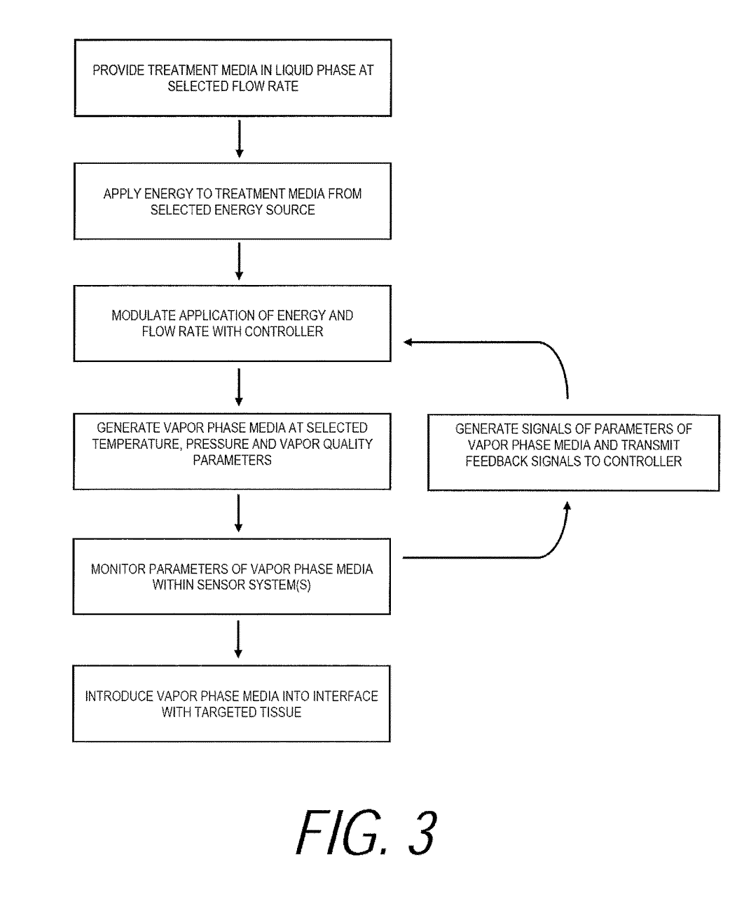

Now turning to FIGS. 2 and 3, the controller 150 is capable of all operational parameters of system 100, including modulating the operational parameters in response to preset values or in response to feedback signals FS from sensor system(s) 175 within the system 100 and probe working end 110. In one embodiment, as depicted in the block diagram of FIG. 3, the system 100 and controller 150 are capable of providing or modulating an operational parameter comprising a flow rate of liquid phase treatment media 122 from pressurized source 120, wherein the flow rate is within a range from about 0.001 to 20 ml/min, 0.010 to 10 ml/min or 0.050 to 5 ml/min. The system 100 and controller 150 are further capable of providing or modulating another operational parameter comprising the inflow pressure of liquid phase treatment media 121 in a range from 0.5 to 1000 psi, 5 to 500 psi, or to 200 psi. The system 100 and controller 150 are further capable of providing or modulating another operational parameter comprising a selected level of energy capable of converting the liquid phase media into a non-liquid, non-ionized gas phase media, wherein the energy level is within a range of about 5 to 2,500 watts; 10 to 1,000 watts or 25 to 500 watts. The system 100 and controller 150 are capable of applying the selected level of energy to provide the phase conversion in the treatment media over an interval ranging from 0.1 second to 10 minutes; 0.5 seconds to 5 minutes, and 1 second to 60 seconds. The system 100 and controller 150 are further capable of controlling parameters of the vapor phase media including the flow rate of non-ionized vapor media proximate an outlet 125, the pressure of vapor media 122 at the outlet, the temperature or mass average temperature of the vapor media, and the quality of vapor media as will be described further below.

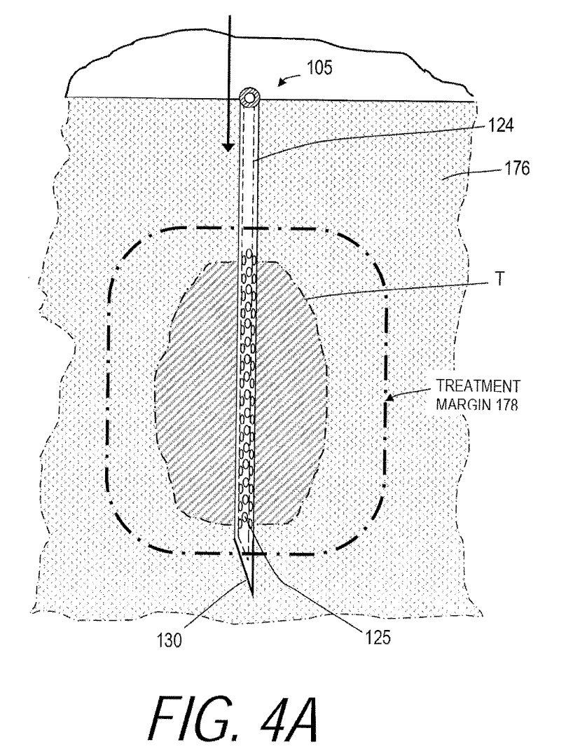

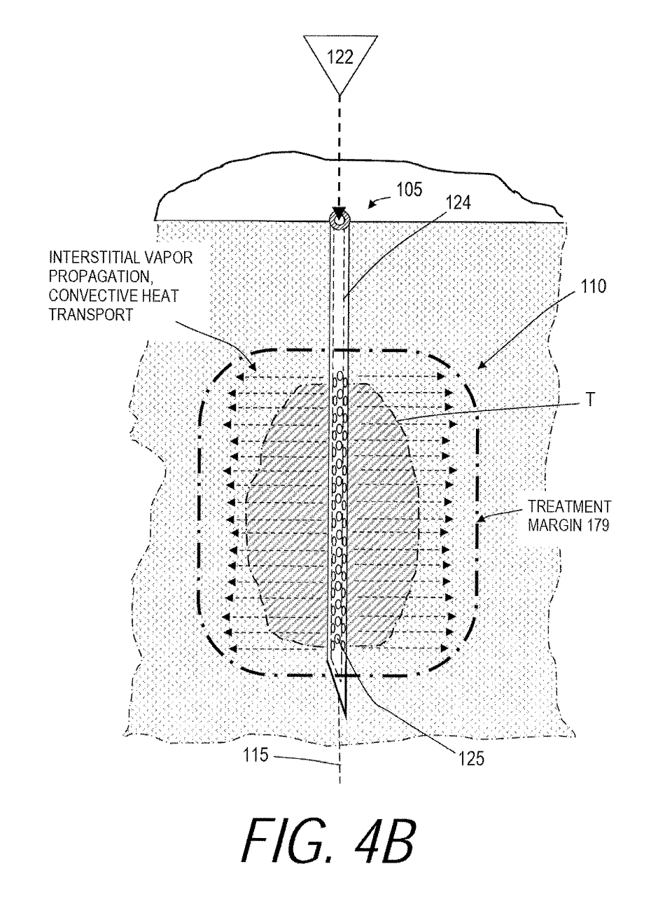

FIGS. 4A and 4B illustrate a working end 110 of the system 100 of FIG. 2 and a method of use. As can be seen in FIG. 4A, a working end 110 is singular and configured as a needle-like device for penetrating into and/or through a targeted tissue T such as a tumor in a tissue volume 176. The tumor can be benign, malignant, hyperplastic or hypertrophic tissue, for example, in a patient's breast, uterus, lung, liver, kidney, gall bladder, stomach, pancreas, colon, GI tract, bladder, prostate, bone, vertebra, eye, brain or other tissue. In one embodiment of the invention, the extension portion 104 is made of a metal, for example, stainless steel. Alternatively or additionally, at least some portions of the extension portion can be fabricated of a polymer material such as PEEK, PTFE, Nylon or polypropylene. Also optionally, one or more components of the extension portion are formed of coated metal, for example, a coating with Teflon® to reduce friction upon insertion and to prevent tissue sticking following use. In one embodiment at in FIG. 4A, the working end 110 includes a plurality of outlets 125 that allow vapor media to be ejected in all radial directions over a selected treatment length of the working end. In another embodiment, the plurality of outlets can be symmetric or asymmetric axially or angularly about the working end 110.

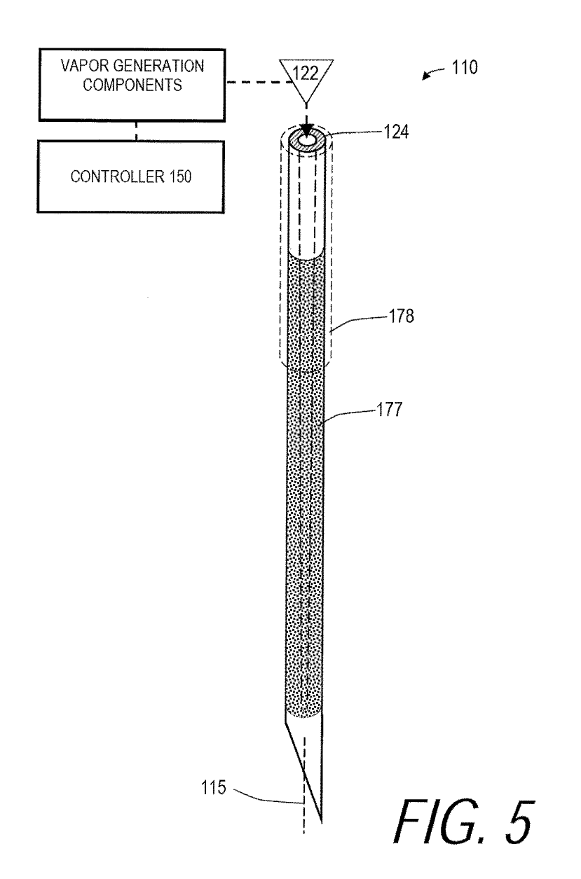

In one embodiment, the outer diameter of extension portion 105 or working end 110 is, for example, 0.2 mm, 0.5 mm, 1 mm, 2 mm, 5 mm or an intermediate, smaller or larger diameter. Optionally, the outlets can comprise microporosities 177 in a porous material as illustrated in FIG. 5 for diffusion and distribution of vapor media flows about the surface of the working end. In one such embodiment, such porosities provide a greater restriction to vapor media outflows than adjacent targeted tissue, which can vary greatly in vapor permeability. In this case, such microporosities insure that vapor media outflows will occur substantially uniformly over the surface of the working end. Optionally, the wall thickness of the working end 110 is from 0.05 to 0.5 mm. Optionally, the wall thickness decreases or increases towards the distal sharp tip 130 (FIG. 5). In one embodiment, the dimensions and orientations of outlets 125 are selected to diffuse and/or direct vapor media propagation into targeted tissue T and more particularly to direct vapor media into all targeted tissue to cause extracellular vapor propagation and thus convective heating of the target tissue as indicated in FIG. 4B. As shown in FIGS. 4A-4B, the shape of the outlets 125 can vary, for example, round, ellipsoid, rectangular, radially and/or axially symmetric or asymmetric. As shown in FIG. 5, a sleeve 178 can be advanced or retracted relative to the outlets 125 to provide a selected exposure of such outlets to provide vapor injection over a selected length of the working end 110. Optionally, the outlets can be oriented in various ways, for example so that vapor media 122 is ejected perpendicular to a surface of working end 110, or ejected is at an angle relative to the axis 115 or angled relative to a plane perpendicular to the axis. Optionally, the outlets can be disposed on a selected side or within a selected axial portion of working end, wherein rotation or axial movement of the working end will direct vapor propagation and energy delivery in a selected direction. In another embodiment, the working end 110 can be disposed in a secondary outer sleeve that has apertures in a particular side thereof for angular/axial movement in targeted tissue for directing vapor flows into the tissue.

FIG. 4B illustrates the working end 110 of system 100 ejecting vapor media from the working end under selected operating parameters, for example a selected pressure, vapor temperature, vapor quantity, vapor quality and duration of flow. The duration of flow can be a selected pre-set or the hyperechoic aspect of the vapor flow can be imaged by means of ultrasound to allow the termination of vapor flows by observation of the vapor plume relative to targeted tissue T. As depicted schematically in FIG. 4B, the vapor can propagate extracellularly in soft tissue to provide intense convective heating as the vapor collapses into water droplets which results in effective tissue ablation and cell death. As further depicted in FIG. 4B, the tissue is treated to provide an effective treatment margin 179 around a targeted tumorous volume. The vapor delivery step is continuous or can be repeated at a high repetition rate to cause a pulsed form of convective heating and thermal energy delivery to the targeted tissue. The repetition rate vapor flows can vary, for example with flow durations intervals from 0.01 to 20 seconds and intermediate off intervals from 0.01 to 5 seconds or intermediate, larger or smaller intervals.

In an exemplary embodiment as shown in FIGS. 4A-4B, the extension portion 105 can be a unitary member such as a needle. In another embodiment, the extension portion 105 or working end 110 can be a detachable flexible body or rigid body, for example of any type selected by a user with outlet sizes and orientations for a particular procedure with the working end attached by threads or Luer fitting to a more proximal portion of probe 102.

In other embodiments, the working end 110 can comprise needles with terminal outlets or side outlets as shown in FIGS. 6A-6B. The needle of FIGS. 6A and 6B can comprise a retractable needle as shown in FIG. 6C capable of retraction into probe or sheath 180 for navigation of the probe through a body passageway or for blocking a portion of the vapor outlets 125 to control the geometry of the vapor-tissue interface. In another embodiment shown in FIG. 6D, the working end 110 can have multiple retractable needles that are of a shape memory material. In another embodiment as depicted in FIG. 6E, the working end 110 can have at least one deflectable and retractable needle that deflects relative to an axis of the probe 180 when advanced from the probe. In another embodiment, the working end 110 as shown in FIGS. 6F-6G can comprise a dual sleeve assembly wherein vapor-carrying inner sleeve 181 rotates within outer sleeve 182 and wherein outlets in the inner sleeve 181 only register with outlets 125 in outer sleeve 182 at selected angles of relative rotation to allow vapor to exit the outlets. This assembly thus provides for a method of pulsed vapor application from outlets in the working end. The rotation can be from about 1 rpm to 1000 rpm.

In another embodiment of FIG. 6H, the working end 110 has a heat applicator surface with at least one vapor outlet 125 and at least one expandable member 183 such as a balloon for positioning the heat applicator surface against targeted tissue, In another embodiment of FIG. 6I, the working end can be a flexible material that is deflectable by a pull-wire as is known in the art. The embodiments of FIGS. 6H and 6I have configurations for use in treating atrial fibrillation, for example in pulmonary vein ablation.

In another embodiment of FIG. 6J, the working end 110 includes additional optional heat applicator means which can comprise a mono-polar electrode cooperating with a ground pad or bi-polar electrodes 184a and 184b for applying energy to tissue. In FIG. 6K, the working end 110 includes resistive heating element 187 for applying energy to tissue. FIG. 6L depicts a snare for capturing tissue to be treated with vapor and FIG. 6M illustrates a clamp or jaw structure. The working end 110 of FIG. 6M includes means actuatable from the handle for operating the jaws.

Sensors for Vapor Flows, Temperature, Pressure, Quality

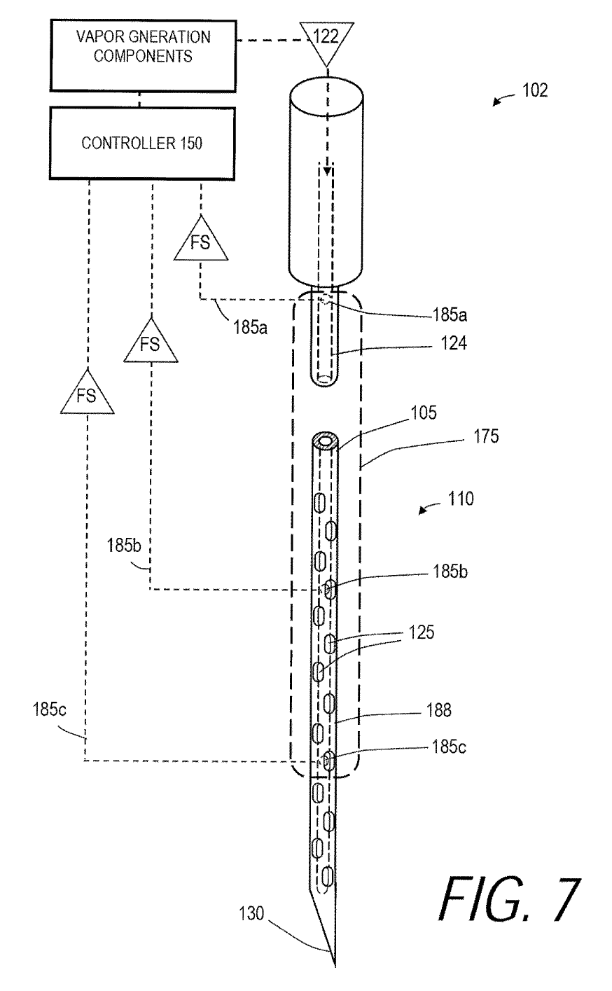

Referring to FIG. 7, one embodiment of sensor system 175 is shown that is carried by working end 110 of the probe 102 depicted in FIG. 2 for determining a first vapor media flow parameter, which can consist of determining whether the vapor flow is in an “on” or “off” operating mode. The working end 110 of FIG. 7 comprises a sharp-tipped needle suited for needle ablation of any neoplasia or tumor tissue, such as a benign or malignant tumor as described previously, but can also be any other form of vapor delivery tool. The needle can be any suitable gauge and in one embodiment has a plurality of vapor outlets 125. In a typical treatment of targeted tissue, it is important to provide a sensor and feedback signal indicating whether there is a flow, or leakage, of vapor media 122 following treatment or in advance of treatment when the system is in “off” mode. Similarly, it is important to provide a feedback signal indicating a flow of vapor media 122 when the system is in “on” mode. In the embodiment of FIG. 7, the sensor comprises at least one thermocouple or other temperature sensor indicated at 185a, 185b and 185c that are coupled to leads (indicated schematically at 186a, 186b and 186c) for sending feedback signals to controller 150. The temperature sensor can be a singular component or can be plurality of components spaced apart over any selected portion of the probe and working end. In one embodiment, a feedback signal of any selected temperature from any thermocouple in the range of the heat of vaporization of treatment media 122 would indicate that flow of vapor media, or the lack of such a signal would indicate the lack of a flow of vapor media. The sensors can be spaced apart by at least 0.05 mm, 1 mm, 5 mm, 10 mm and 50 mm. In other embodiments, multiple temperature sensing event can be averaged over time, averaged between spaced apart sensors, the rate of change of temperatures can be measured and the like. In one embodiment, the leads 186a, 186b and 186c are carried in an insulative layer of wall 188 of the extension member 105. The insulative layer of wall 188 can include any suitable polymer or ceramic for providing thermal insulation. In one embodiment, the exterior of the working end also is also provided with a lubricious material such as Teflon® which further insures against any tissue sticking to the working end 110.

Still referring to FIG. 7, a sensor system 175 can provide a different type of feedback signal FS to indicate a flow rate or vapor media based on a plurality of temperature sensors spaced apart within flow channel 124. In one embodiment, the controller 150 includes algorithms capable of receiving feedback signals FS from at least first and second thermocouples (e.g., 185a and 185c) at very high data acquisition speeds and compare the difference in temperatures at the spaced apart locations. The measured temperature difference, when further combined with the time interval following the initiation of vapor media flows, can be compared against a library to thereby indicate the flow rate.

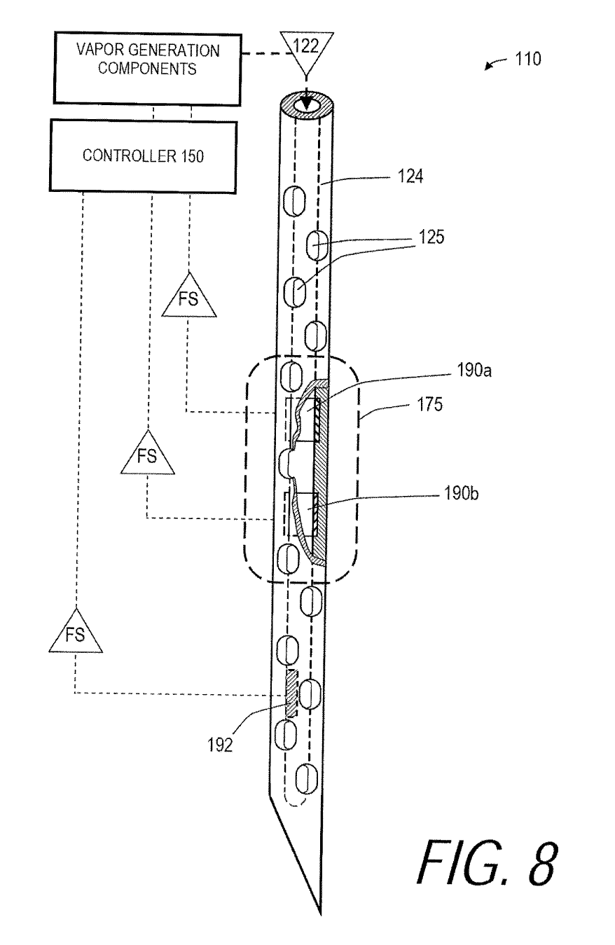

Another embodiment of sensor system 175 in a similar working end 110 is depicted in FIG. 8, wherein the sensor is configured for indicating vapor quality—in this case based on a plurality of spaced apart electrodes 190a and 190b coupled to controller 150 and an electrical source (not shown). In this embodiment, a current flow is provided within a circuit to the spaced apart electrodes 190a and 190b and during vapor flows within channel 124 the impedance will vary depending on the vapor quality or saturation, which can be processed by algorithms in controller 150 and can be compared to a library of impedance levels, flow rates and the like to thereby determine vapor quality. It is important to have a sensor to provide feedback of vapor quality, which determines how much energy is being carried by a vapor flow. The term “vapor quality” is herein used to describe the percentage of the flow that is actually water vapor as opposed to water droplets that is not phase-changed. In another embodiment (not shown) an optical sensor can be used to determine vapor quality wherein a light emitter and receiver can determine vapor quality based on transmissibility or reflectance of a vapor flow.

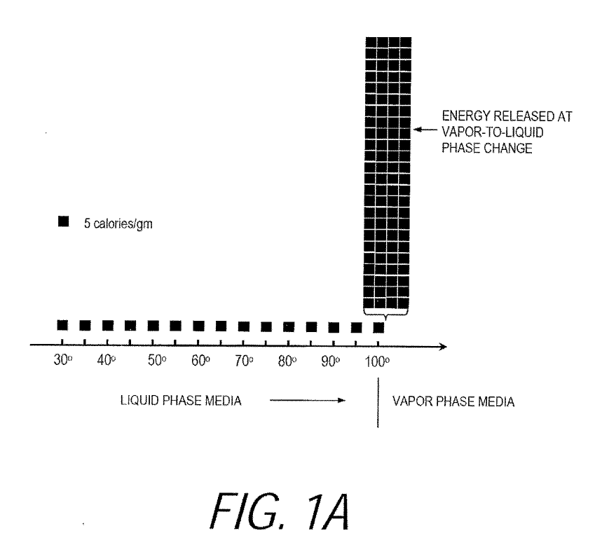

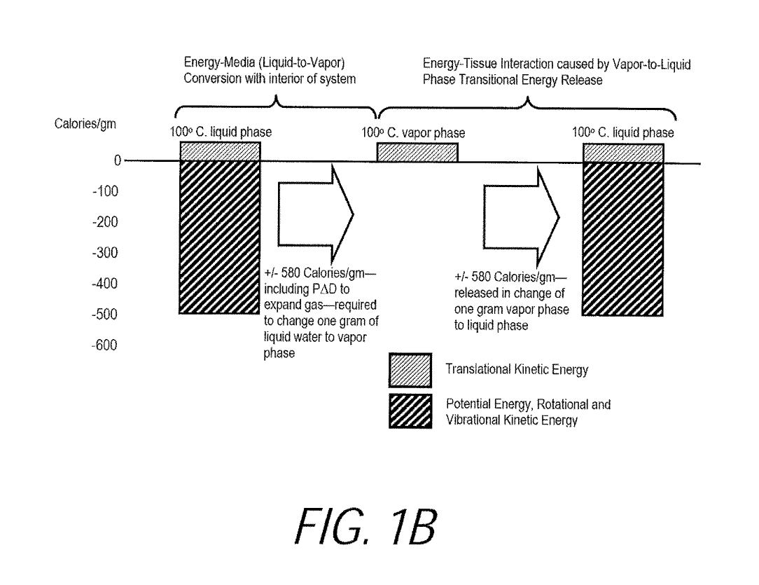

FIG. 8 further depicts a pressure sensor 192 in the working end 110 for providing a signal as to vapor pressure. In operation, the controller can receive the feedback signals FS relating to temperature, pressure and vapor quality to thereby modulate all other operating parameters described above to optimize flow parameters for a particular treatment of a target tissue, as depicted in FIG. 1. In one embodiment, a MEMS pressure transducer is used, which are known in the art. In another embodiment, a MEMS accelerometer coupled to a slightly translatable coating can be utilized to generate a signal of changes in flow rate, or a MEMS microphone can be used to compare against a library of acoustic vibrations to generate a signal of flow rates.

Inductive Vapor Generation Systems

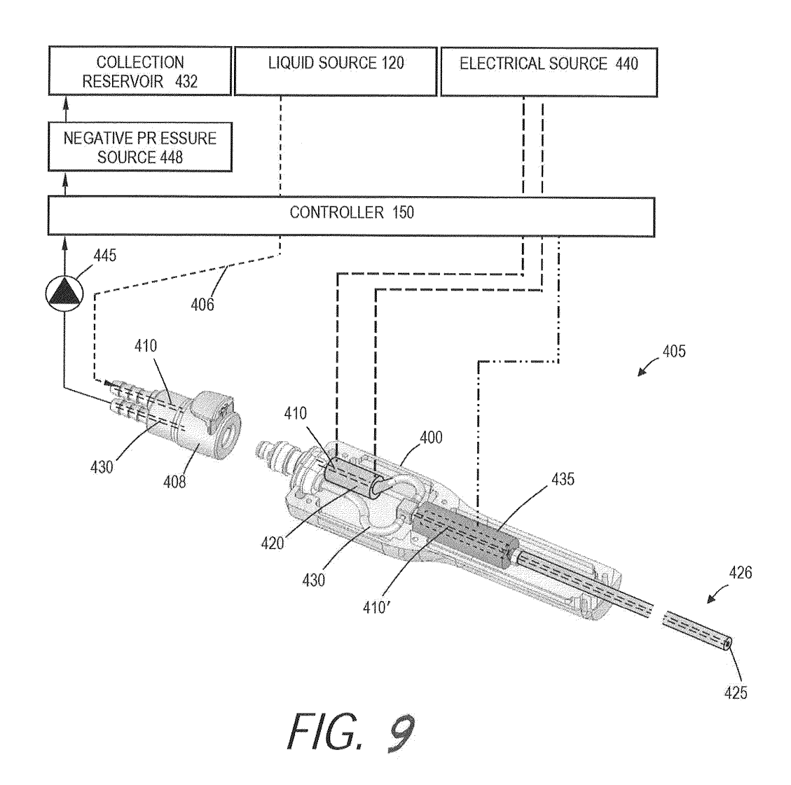

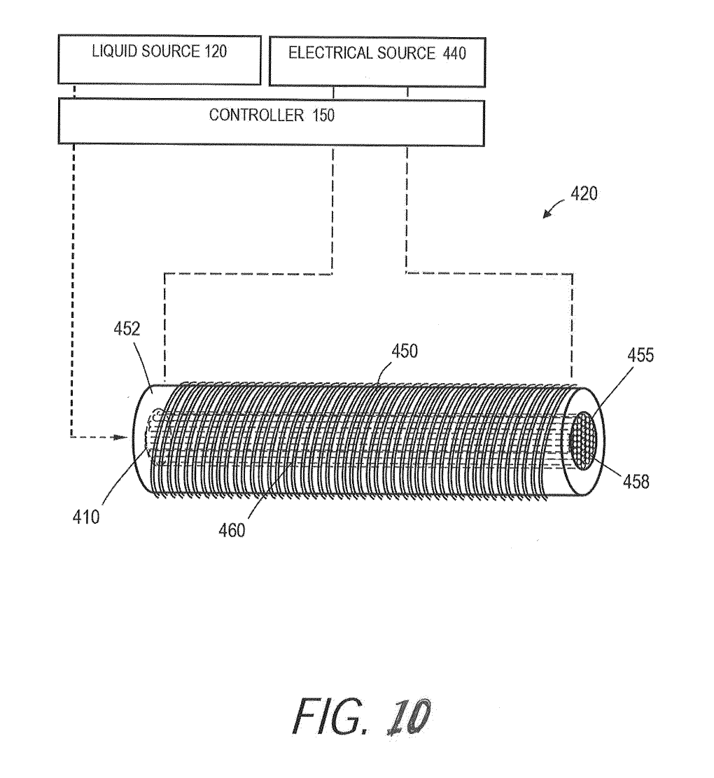

FIGS. 9 and 10 depict a vapor generation component that utilizes and an inductive heating system within a handle portion 400 of the probe or vapor delivery tool 405. In FIG. 9, it can be seen that a pressurized source of liquid media 120 (e.g., water or saline) is coupled by conduit 406 to a quick-connect fitting 408 to deliver liquid into a flow channel 410 extending through an inductive heater 420 in probe handle 400 to at least one outlet 425 in the working end 426. In one embodiment shown in FIG. 9, the flow channel 410 has a bypass or recirculation channel portion 430 in the handle or working end 426 that can direct vapor flows to a collection reservoir 432. In operation, a valve 435 in the flow channel 410 thus can direct vapor generated by inductive heater 420 to either flow channel portion 410′ or the recirculation channel portion 430. In the embodiment of FIG. 10, the recirculation channel portion 430 also is a part of the quick-connect fitting 408.

In FIG. 9, it can be seen that the system includes a computer controller 150 that controls (i) the electromagnetic energy source 440 coupled to inductive heater 420, (ii) the valve 435 which can be an electrically-operated solenoid, (iii) an optional valve 445 in the recirculation channel 430 that can operate in unison with valve 435, and (iv) optional negative pressure source 448 operatively coupled to the e recirculation channel 430.

In general, the system of the invention provides a small handheld device including an assembly that utilized electromagnetic induction to turn a sterile water flow into superheated or dry vapor which can is propagated from at least one outlet in a vapor delivery tool to interface with tissue and thus ablate tissue. In one aspect of the invention, an electrically-conducting microchannel structure or other flow-permeable structure is provided and an inductive coil causes electric current flows in the structure. Eddies within the current create magnetic fields, and the magnetic fields oppose the change of the main field thus raising electrical resistance and resulting in instant heating of the microchannel or other flow-permeable structure. In another aspect of the invention, it has been found that corrosion-resistant microtubes of low magnetic 316 SS are best suited for the application, or a sintered microchannel structure of similar material. While magnetic materials can improve the induction heating of a metal because of ferromagnetic hysteresis, such magnetic materials (e.g. carbon steel) are susceptible to corrosion and are not optimal for generating vapor used to ablate tissue. In certain embodiments, the electromagnetic energy source 440 is adapted for inductive heating of a microchannel structure with a frequency in the range of 50 kHz to 2 Mhz, and more preferably in the range of 400 kHz to 500 kHz. While a microchannel structure is described in more detail below, it should be appreciated that the scope of the invention includes flow-permeable conductive structures selected from the group of woven filaments structures, braided filament structures, knit filaments structures, metal wool structures, porous structures, honeycomb structure and an open cell structures.

In general, a method of the invention comprises utilizing an inductive heater 420 of FIGS. 9-10 to instantly vaporize a treatment media such as deionized water that is injected into the heater at a flow rate of ranging from 0.001 to 20 ml/min, 0.010 to 10 ml/min, 0.050 to 5 ml/min., and to eject the resulting vapor into body structure to ablate tissue. The method further comprises providing an inductive heater 420 configured for a disposable hand-held device (see FIG. 9) that is capable of generating a minimum water vapor that is at least 70% water vapor, 80% water vapor and 90% water vapor.

FIG. 10 is an enlarged schematic view of inductive heater 420 which includes at least one winding of inductive coil 450 wound about an insulative sleeve 452. The coil 450 is typically wound about a rigid insulative member, but also can comprise a plurality of rigid coil portions about a flexible insulator or a flexible coil about a flexible insulative sleeve. The coil can be in handle portion of a probe or in a working end of a probe such as a catheter. The inductive coil can extends in length at least 5 mm, 10 mm, 25 mm, 50 mm or 100 m.

In one embodiment shown schematically in FIG. 10, the inductive heater 420 has a flow channel 410 in the center of insulative sleeve 452 wherein the flows passes through an inductively heatable microchannel structure indicated at 455. The microchannel structure 455 comprises an assembly of metal hypotubes 458, for example consisting of thin-wall biocompatible stainless steel tube tightly packed in bore 460 of the assembly. The coil 450 can thereby inductively heat the metal walls of the microchannel structure 455 and the very large surface area of structure 455 in contact with the flow can instantly vaporize the flowable media pushed into the flow channel 410. In one embodiment, a ceramic insulative sleeve 452 has a length of 1.5″ and outer diameter of 0.25″ with a 0.104″ diameter bore 460 therein. A total of thirty-two 316 stainless steel tubes 458 with 0.016″ O.D., 0.010″ I.D., and 0.003″ wall are disposed in bore 460. The coil 450 has a length of 1.0″ and comprises a single winding of 0.026″ diameter tin-coated copper strand wire (optionally with ceramic or Teflon® insulation), and can be wound in a machined helical groove in the insulative sleeve 452. A 200 W RF power source 440 is used operating at 400 kHz with a pure sine wave. A pressurized sterile water source 120 comprises a computer controlled syringe that provides fluid flows of deionized water at a rate of 3 ml/min which can be instantly vaporized by the inductive heater 420. At the vapor exit outlet or outlets 125 in a working end, it has been found that various pressures are needed for various tissues and body cavities for optimal ablations, ranging from about 0.1 to 20 psi for ablating body cavities or lumens and about 0.1 psi to 100 psi for interstitial ablations.

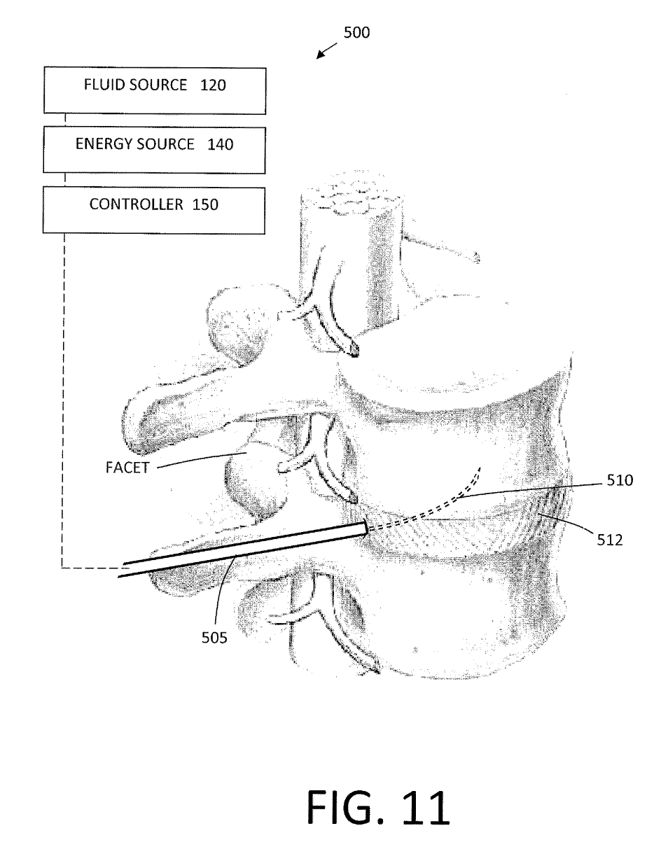

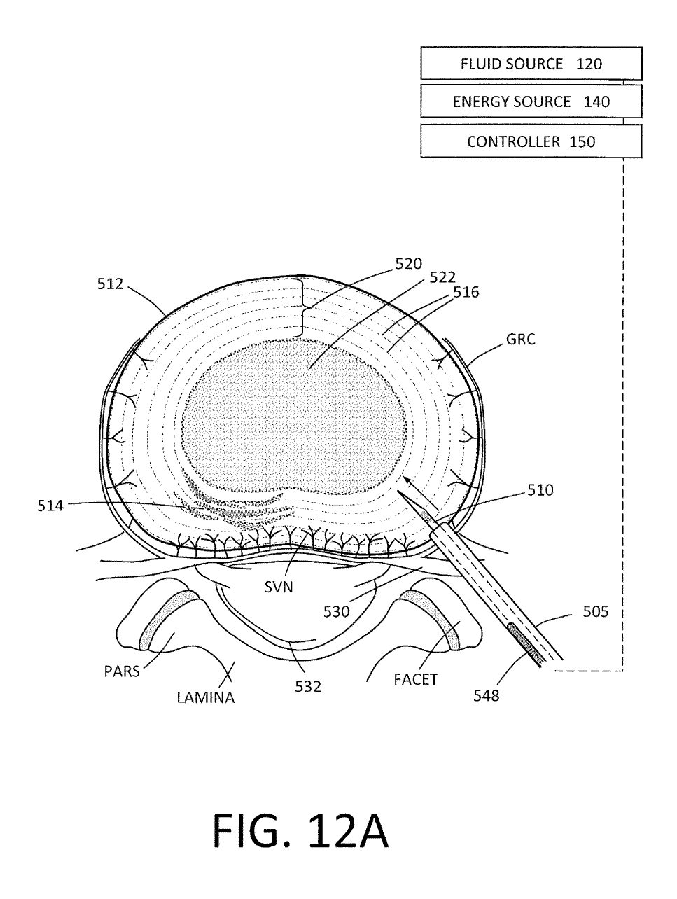

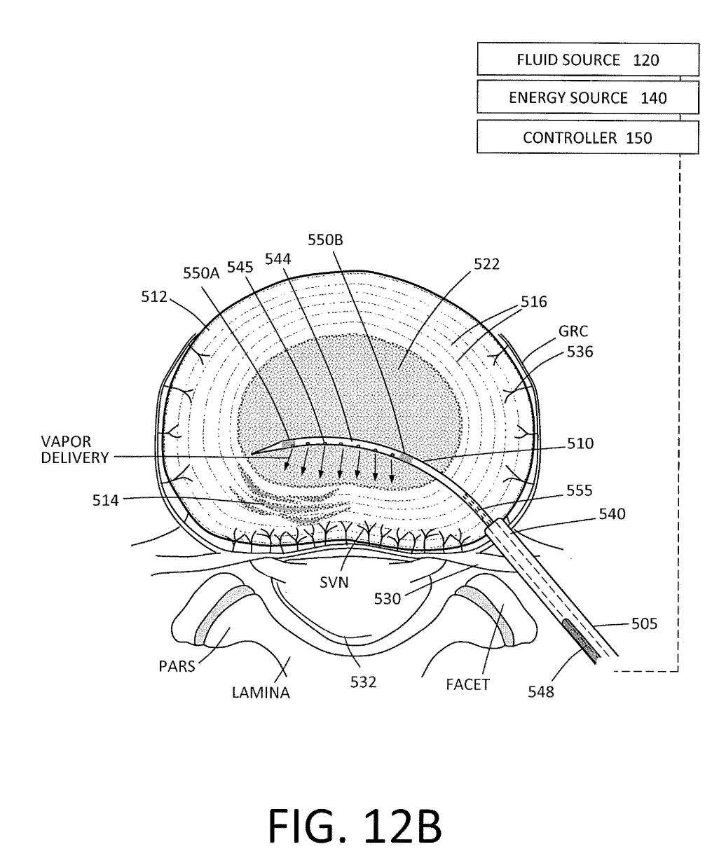

FIGS. 11 and 12A-12B schematically depict another system 500, vapor delivery tool with an elongated introducer 505 with bore 508 therein carrying an extendable vapor delivery needle or extension member 510, and method of use configured for treating a back pain, and more particularly in one embodiment for treating a patient's disc to alleviate discogenic pain.

It has been reported that 80% of U.S. adults suffer from lower back pain at some point in their lives. In many cases, the pain is related to a disc disorder such as an internal disc disruption, a bulging disc or a herniated disc. While many people are asymptomatic people with a disc bulging or internal disc disruption, about 40% of chronic back pain patients will have tears or disruptions within their discs that are often can be invisible on MRI.

FIG. 12A is a schematic view of an intervertebral disc 512 with internal disc disruption consisting of concentric tears 514 within the lamellae 516 of the annulus fibrosus 520. It is believed that such internal disc disruptions are a major cause of discogenic pain.

The annulus fibrosis 520 is the tough circular exterior of the intervertebral disc 512 that surrounds the nucleus pulposus 522. The annulus is a layered structure, in that it contains 15 to 25 sheets of collagen called lamellae 516. The annulus is predominantly formed of collagen fibers which have a much lower water content than the nucleus 522. The annulus 520 securely connects the vertebral bodies above and below the disc, and further provides containment of the highly pressurized nucleus 522 and protects the nerve-laden outer one-third of the annulus and posterior epidural neural structures, e.g., the delicate nerve roots 530 and thecal sac 532.

The nucleus pulposus 522 is a gelatinous-like material in the core of the vertebral disc. In a young, healthy patient, the nucleus 522 has a gelatinous material with high water content, comprising mostly proteoglycans produced by the cells of the nucleus. The elastic inner structure allows the vertebral disc 512 to withstand forces of compression and torsion. With age, the body's discs dehydrate and become stiffer, causing the disc to be less able to adjust to compression.

Referring to FIG. 12A, due to the higher pressure within the nucleus pulposus 522, irritating nuclear material can migrate from the nucleus through the tear 514 outwardly to contact the sinuvertebral nerves SVN that lie within the outer one-third of the annulus 520. Such leakage of nuclear material can cause in many patients an inflammatory reaction within the outer disc portion that causes chronic and debilitating back and/or leg pain. Nerves 536 in the disc that branch from the sympathetic nervous system or grey ramus communicans GRC also can be irritated by leaking nuclear material (FIG. 12A).

Referring to FIG. 11, the system 500 includes a tool with a handle as shown in FIG. 2 with a liquid media source 120, energy source 140 and controller 150 that are adapted to produce and deliver a high temperature vapor media through an elongated vapor delivery tool or extension member 510. The system embodiment 500 of FIG. 11 is similar to that of FIG. 6C, wherein the vapor delivery needle or extension member is extendable from bore 508 in introducer sleeve 505.

Referring to FIGS. 11 and 12A, the physician advances the introducer 505 through a skin incision to a targeted location on the disc 512. The distal end 540 of the elongated sleeve-type introducer 505 has a dull tip, with optional radiopaque markings (not shown) for viewing under fluoroscopy, which is pressed against the wall of disc 512 in the selected location. As further can be seen in be FIG. 12A, the vapor-delivery needle 510 then can be advanced from the introducer 505 into the annulus 520. The angle and orientation of the introducer 505 can be determined by the location of targeted treatment in disc, as well as by selection of the vapor delivery needle 510.

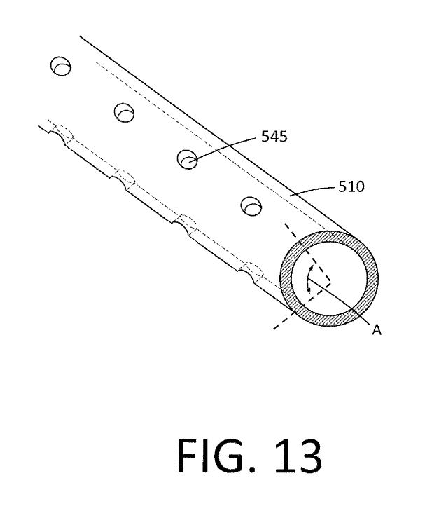

Now turning to FIG. 12B, one embodiment of vapor delivery tool or needle 510 comprises a metal needle of a shape memory material such as Nitinol that has a predetermined curved portion 544 that permits a plurality of vapor outlets 545 to be positioned to face posteriorly for treating a disc defect in a posterior portion of the disc 512. In the embodiment of FIG. 12B, it can be understood that the curved vapor delivery needle 510 is rotationally keyed with handle (not shown) and/or introducer 505 and a visual marking 548 indicates to the physician the direction that the needle will curve when in the interior of the disc 512. The curved portion 544 or working end further is configured with radiopaque markers 550A and 550B for positioning the working end under fluoroscopy. In FIG. 12B, the needle 510 is advanced to a suitable location to deliver vapor adjacent the target tissue which in this case is the posterior disc region, wherein vapor delivery is indicated by arrows. In the embodiment of FIG. 12B, the vapor is delivered from a plurality of vapor outlets 545 that all face either the same direction or are configured to emit vapor within a radial arc A of less than 90° as shown in FIG. 13, or optionally less than 45°.

In use, one method comprises delivering a high quality water vapor, for example at least 70% water vapor, at least 80% water vapor or at least 90% water vapor for between 1 second and 30 seconds at a pressure ranging from 0.01 psi to 20 psi in the interior bore 555 of the needle 510. A method of the invention comprises permitting condensation of the water vapor in the targeted site, wherein the applied energy can range from 10 J to 1,000 J or more. Of particular interest, the vapor can find a path through the tear 514 in annulus 520 and ablate nerve receptors of the sinuvertebral nerves SVN as shown in FIG. 12B. This modality of energy application via convective heating (non-desiccating and entirely without the potential of tissue carbonization) is far different from IDET and laser discectoiny modalities—which must rely on thermal diffusion from an electrode or optic fiber to ablate nerve receptors and may not apply energy rapidly enough to damage nerve receptors without diffusing heat too far outwardly into the disc periphery, the vertebral endplates and adjacent tissues.

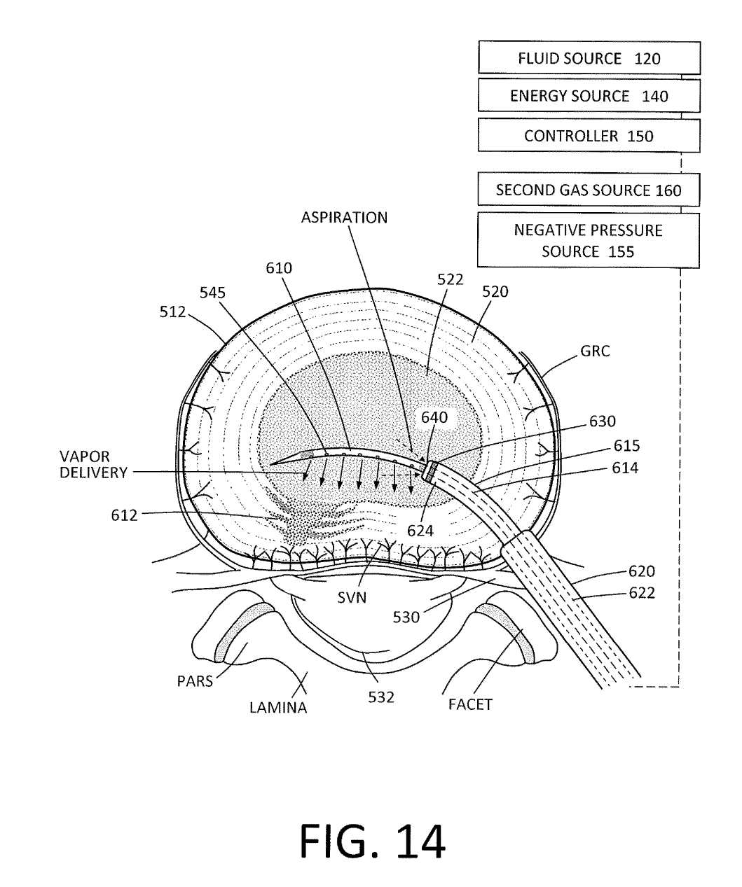

FIG. 14 depicts another embodiment 600 of vapor delivery system that includes additional subsystems as described in text accompanying the embodiment of FIG. 2. The system again includes a liquid media source 120, energy source 140 and controller 150 for generating and expelling a therapeutic heated vapor media from the vapor delivery needle 610. This system embodiment 600 of FIG. 14 further includes a negative pressure source 155 and an optional second gas source 160 for mixing with the heated vapor, as will be described below. In the method depicted in FIG. 14, the defect in the disc 512 consists of a radially annular tear 612 and disc nucleus protrusion.

In the embodiment of FIG. 14, it can be seen that the vapor delivery needle 610 is extendable from lumen 614 in sleeve 615. The sleeve 615 is coupled to negative pressure source 155 and thus comprises an aspiration sleeve to allow negative pressures to subtract pressure, vapor and liquid media from the interior of the disc 512. The assembly of vapor-delivery needle 610 and sleeve 615 is extendable and retractable relative to introducer sleeve 620 and bore 622 therein.

In FIG. 14, it can be understood that the assembly of needle 610, sleeve 615 and introducer 620 can be advanced through a skin incision into the disc 512. Thereafter, the needle 610 and sleeve 615 can be advanced through a small incision in the disc so that the distal end 624 of sleeve 615 is within the treatment region in the nucleus 522. The handle of the device (not shown) is configured with handle portions to allow axial and/or rotational movement of the needle 610 and sleeve 615. The distal end 624 of sleeve 615 is shown with radiopaque marking 630 to allow the physician to insure the location at which aspiration forces are applied, that is, at the open termination 640 of lumen 614 in sleeve 615.

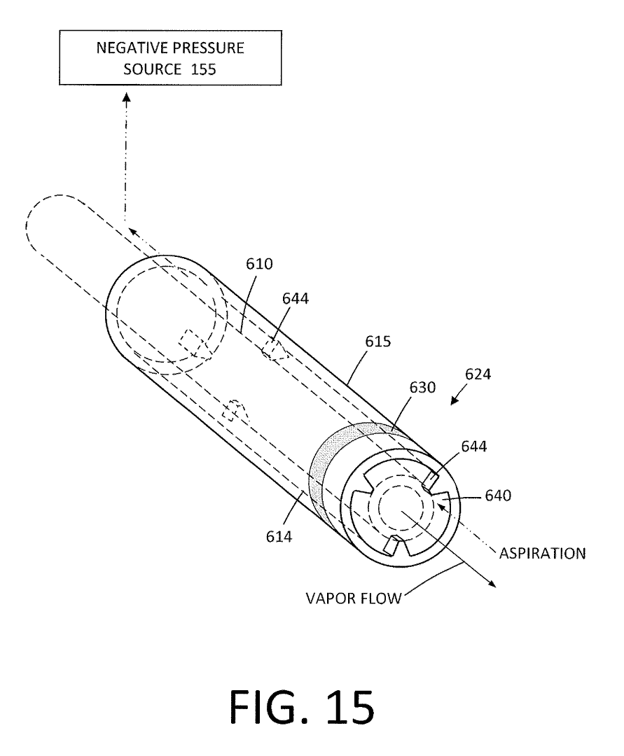

FIG. 15 is an enlarged view of the distal end 624 of sleeve 615 showing the extendable-retractable vapor delivery needle 610. It can be seen that bore 614 in sleeve 615 is larger than the outer diameter of needle 610 to provide an annular space that can function as an aspiration lumen. Thus, negative pressure or aspiration forces can provided at the annular open termination 640 of lumen 614. In one embodiment as shown in FIG. 15, the sleeve 615 is configured with radially spaced apart protrusions 644 at least at the distal end of lumen 614 to maintain the annular lumen 614 in an open configuration. Further, the free space between the vapor needle 610 and the wall of sleeve 615 provides a thermally insulative gap that reduces heating of the sleeve 615 and prevents unwanted condensation of vapor in the vapor delivery needle 610 before the vapor is expelled from ports 545.

In use, the method of vapor delivery is the same as described above to allow vapor propagation and condensation to apply energy to the annular defect 612 while activation of the negative pressure source reduces intradiscal pressure during treatment and further can extract vapor and liquids.

In another method of the invention, still referring to FIG. 14, the second gas source 160 can be actuated contemporaneous with vapor delivery for one or more purposes. In one method, a biocompatible gas such as CO, can be introduced to reduce the mass average temperature of the vapor media. For example, in one embodiment, the combined vapor media can be reduced from the water vapor's temperature of at least 100° C. to a lower mass average temperature of approximately 60° C., or approximately 70° C., or approximately 80° C. In treating disc defects, it is believed that lower temperatures of the mass of the vapor media, such as a maximum of 60° C., 70° C. or 80° C. can ablate nerve receptors and migrate within annular tears to thus eliminate pain, with lesser risk of unwanted thermal diffusion.

In another related method, the second gas source 160 can comprise a source of oxygen and/or ozone. In recent years, it has been found that that oxygen and/or ozone (O₂O₃) injections can be used to treat lower back pain. See, e.g., M. Paoloni, et al., “Intramuscular Oxygen-Ozone Therapy in the Treatment of Acute Back Pain With Lumbar Disc Herniation,” SPINE Vol. 34, No. 13, pp. 1337-1344. Such treatments have used intradiscal or intraforaminal injections, as well a paravertebral intramuscular injections. Such O₂O₃ therapies are known in medicine and are based on the exploitation of the chemical properties of ozone, which is an unstable form of oxygen. In the treatment of disc defects, the O₂O₃ injection is believed to have an effect on proteoglycans within the disc's nucleus pulposus which ultimately reduces nucleus volume and hence reduces intradiscal pressure, and further has and analgesic and anti-inflammatory effect. By combining a thermal vapor with an O₂O₃ component, the resulting vapor can have a lower mass average temperature as well as providing the chemical or pharmacologic effects described above.

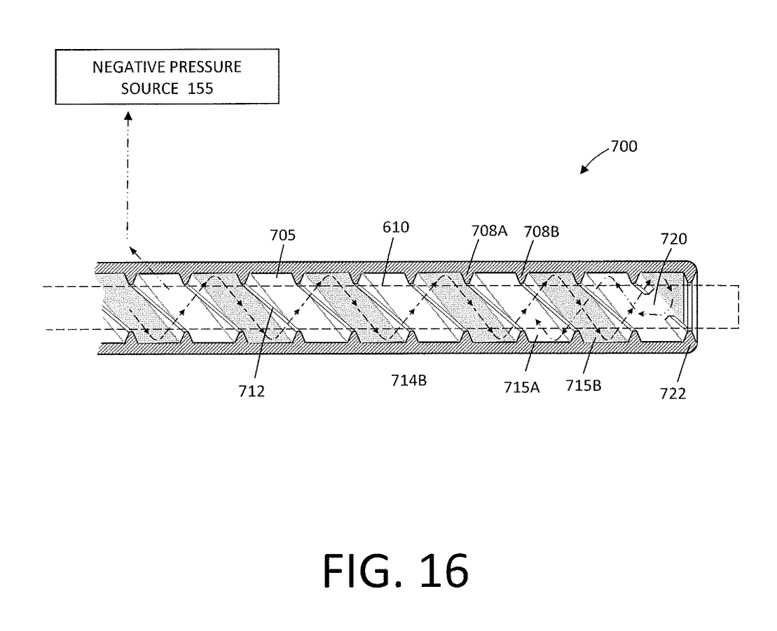

FIG. 16 illustrates another embodiment of the invention, which comprises a sleeve 700 that houses an extendable-retractable vapor delivery needle 610 in phantom view, and for example can comprise the intermediate sleeve of FIG. 14. As can be seen in FIG. 16, the sleeve 700 has lumen 705 with dual-lead helical elements 708A and 708B that have a small edge or surface area 712 that contacts the vapor delivery needle 610. The helical elements 708A and 708B thus form dual, adjacent helical channels 715A and 715B that wind helically around the slidable vapor delivery needle 610. Thus, the space or air gap provided by the channels 715A and 715B provides a thermally insulative gap which can assist in preventing unwanted heating of the sleeve 700. In the embodiment of FIG. 16, the distal termination of channels 715A and 715B have a notch or gap indicated at 720 that allows air or gas flow between the channels when a vapor delivery needle 610 is disposed therein. Further, one of the channels 715A is connected to a negative pressure source 155 at a proximal handle end which thus allow for air or gas flow in a distal direction from the handle (see arrows) and then reverse to flow back toward the negative handle and negative pressure source 155 in the proximal direction. Such a circulating gas flow thus can help in maintaining the sleeve at a low temperature. Further, the negative pressure source and an inlet restrictor or pressure relief valve can provide a negative pressure in channels 715A and 715B to provide further insulative value to the space around the vapor delivery needle. In another embodiment (not shown), the distal end 722 of the sleeve can have an opening to apply negative pressure to the channel 715A to aspirate gas from the treatment site, as well to draw gas through the cooperating channel 715B. It should be appreciated that a gas inflow source connected to channel 715B to cooperate with a negative pressure source 155 coupled to channel 715A. It should be appreciated that the gas flow in channels 715A and 715B can be a cooled gas or cryogenic fluid for providing any desired cooling effect.

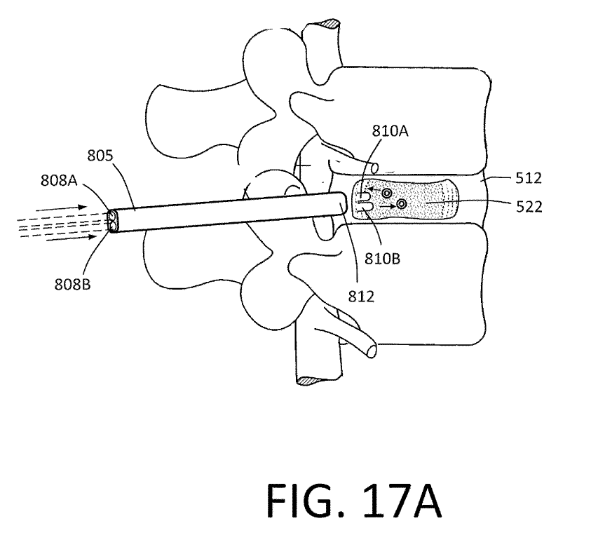

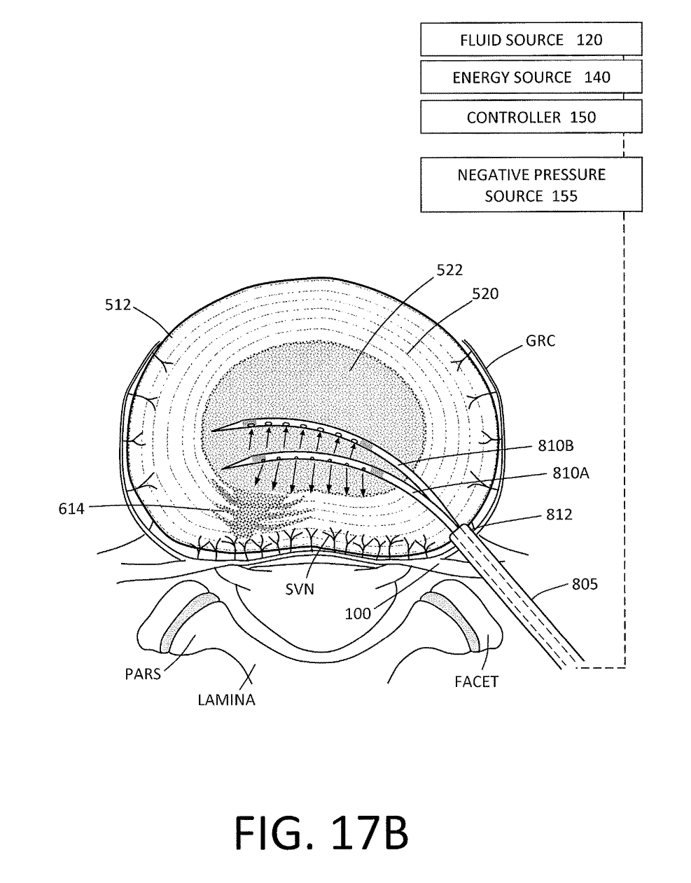

FIG. 17A-17B illustrate another embodiment in which introducer sleeve 805 houses first and second passageways 808A and 808B that carry extendable-retractable extension members or needles 810A and 801B. The cross-section of sleeve 805 can be round, oval or rectangular to accommodate the needles and provide a small cross-sectional dimension for allowing a minimally invasive approach. The distal tip 812 of sleeve 805 is configured to allow both needles to penetrate the disc proximate the engagement of the distal tip 812 with the disc 512. The tip 812 can be angled or beveled to interface with the disc 512 at any anticipated angle of approach to thus allow the needles 810A and 810B to enter the disc, wherein the needles' relationship can be (i) above and below each other, or (ii) side to side with one another. In this embodiment, referring to FIG. 17B, needle 810A is coupled to the vapor source 150 and needle 810B is operatively coupled to the negative pressure source 160. The method of use is similar to that describe above, wherein vapor is introduced to thermally treat the disc tissue and the negative pressure source 160 is used to reduce pressure in the disc nucleus 522 and/or extract fluids. The needles can have a straight shape or any curved memory shape.

It should be appreciated that the method of the invention includes actuating independent vapor injection and aspiration needles that are introduced from a single sleeve or multiple sleeves introduces on one side of a disc or bi-laterally.

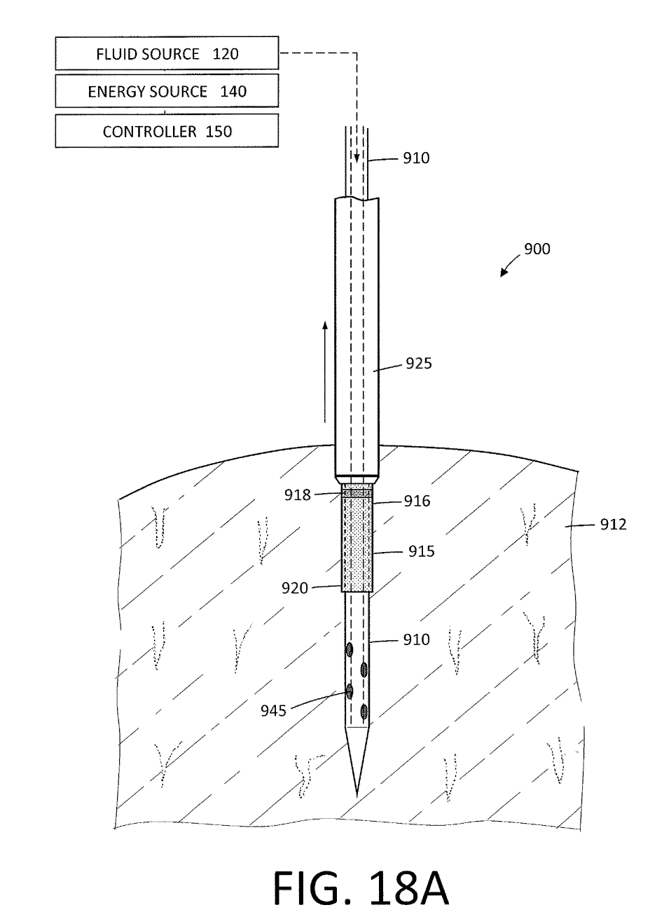

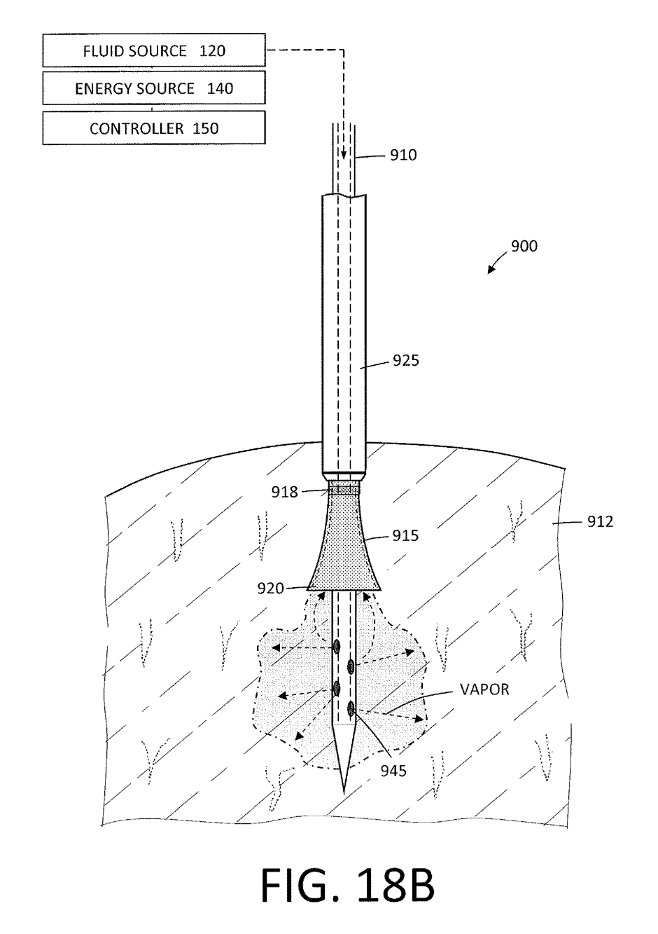

FIGS. 18A-18B depict another embodiment of a working end 900 the invention that comprises means for preventing vapor propagation in an unwanted direction retrograde along the shaft of the vapor needle 910. As can be seen in FIG. 18A, the vapor delivery needle 910 can be in introduced into any tissue 912, such as disc tissue in FIGS. 12A-12B, and the tissue may not have characteristics suitable for sealably pressing against the shaft of needle 910. In the embodiment of FIG. 18A, the needle 910 is provided with a thin-wall, elastomeric sleeve 915 that is bonded in its proximal aspect 916 to the needle shaft along bond line 918. The distal end 920 of the elastomeric sleeve 915 is free and not bonded to the needle shaft. In a method of use, the needle 910 together with outer protective sleeve 925 are inserted into tissue 912 and then the protective sleeve is retracted to expose the elastomeric sleeve 915.

FIG. 18B illustrates the needle 910 in use with vapor (see arrows) being expelled from vapor outlets 945 wherein the vapor propagates outwardly into tissue but also tends to flow retrograde along the needle shaft. As can be seen in FIG. 18B, any tendency of vapor to flow retrograde will be immediately captured by outward expansion and ballooning of the distal end 902 of the elastomeric sleeve 915—thus preventing any further retrograde flow of the vapor.

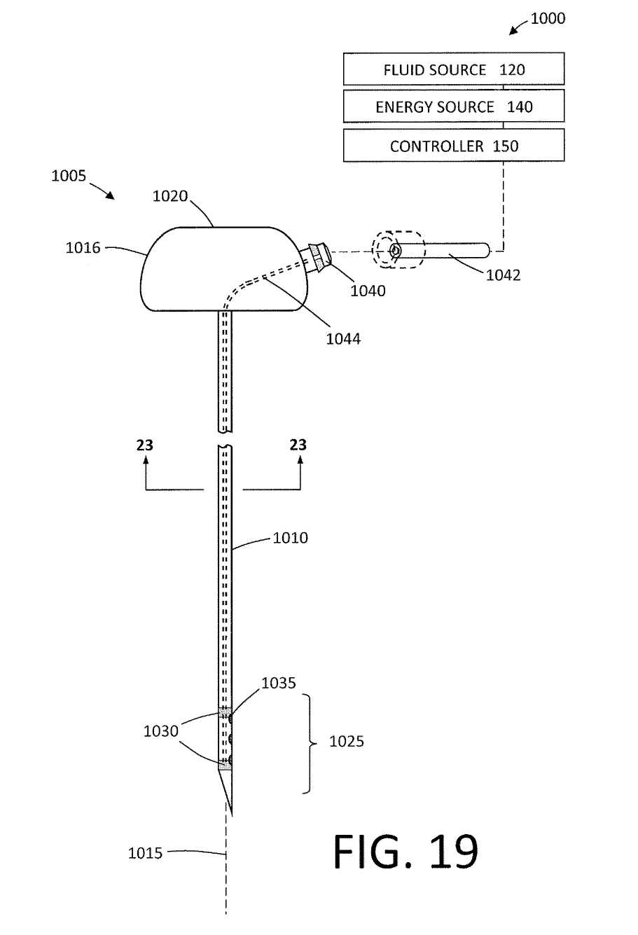

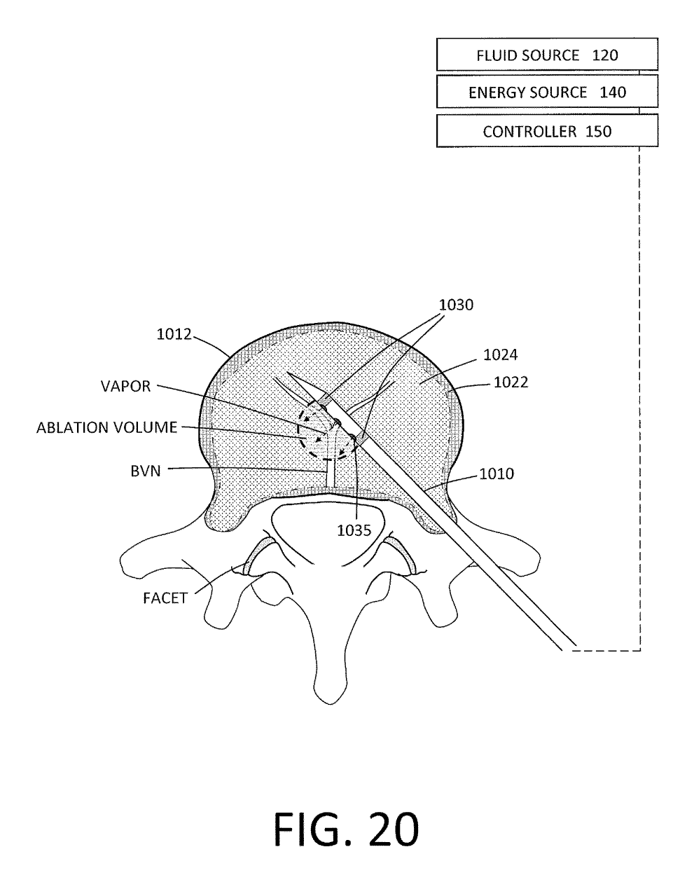

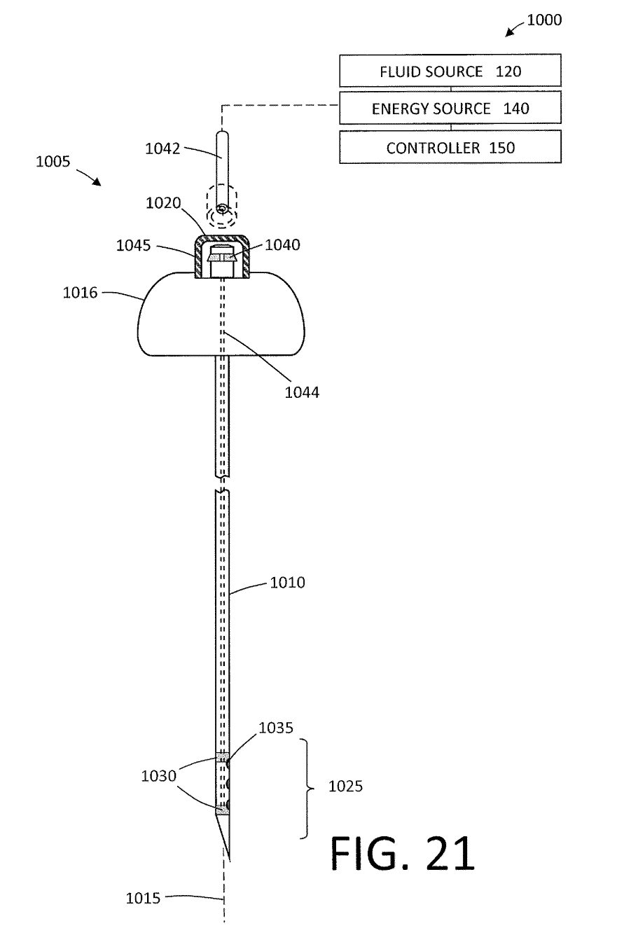

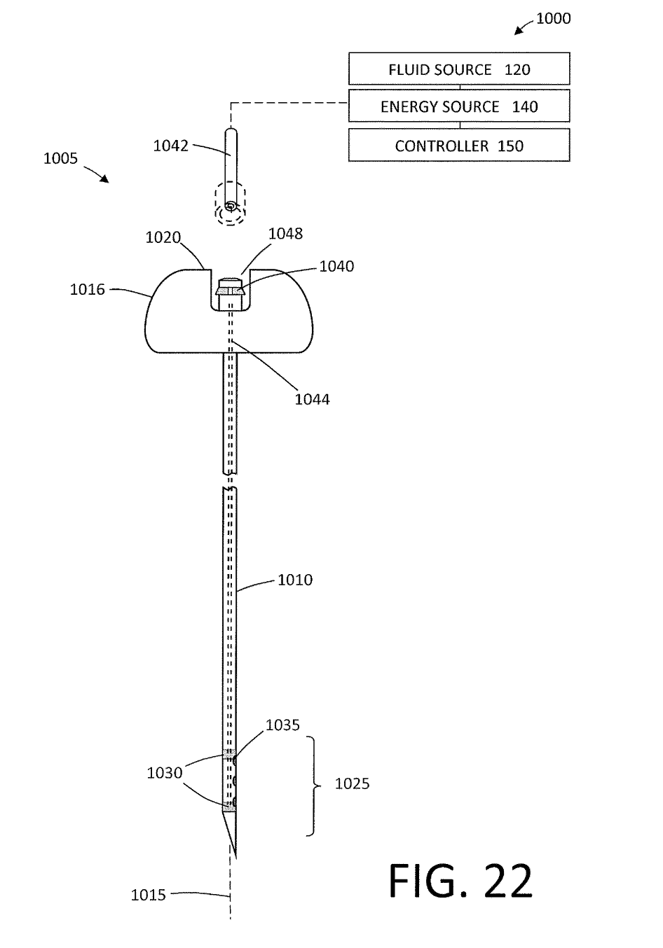

FIGS. 19-20 illustrate another ablation system 1000 and method corresponding to the invention which includes a vapor delivery needle of probe 1005 having an extension portion or shaft 1010 extending along axis 1015 that is configured for driving into bone for ablation of intraosseal nerves, and in one example, a basivertebral nerve BVN in the interior of vertebral body 1016 as shown in FIG. 20. It is believed that certain types of back pain, which differ from types of discogenic pain and vertebral fracture pain, can be alleviated by ablation of nerves within the center of a vertebral body. FIG. 19 illustrates a probe 1005 with a proximal handle 1016 that is configured with hammering surface 1020 to allow driving the sharp-tipped needle shaft 1010 through cortical hone 1022 and cancellous bone 1024 to the location of the targeted nerve. The shaft 1010 can be of a medical grade stainless steel suited for hammering into bone, similar to probes used in vertebroplasty procedures. In one embodiment, the working end 1025 of the needle has radiopaque markings 1030 proximate vapor outlets 1035 that can be oriented on one side of the working end 1025 to orient vapor flow and the ablation in the desired direction, for example toward the center of the vertebral body as shown in FIG. 20. In the embodiment of FIG. 19, the handle 1016 has a fitting 1040 coupling for connecting a flexible vapor delivery tube 1042 to the handle to communicate with vapor delivery lumen 1044 in the probe. The probe of FIG. 19 provides the vapor connection fitting 1040 non-aligned with the needle shaft 1010 or its axis to allow the use of a hammer against hammering surface 1020 after the vapor delivery tube 1042 has been connected the device. In another embodiment of FIG. 21, the proximal handle 1016 can have a fitting 1040 in line with axis 1015 and aligned with shaft 1010 of the device with a removeable or moveable hammering surface 1020 that can cover and protect the fitting 1040. In the embodiment of FIG. 21, a removable cap 1045 carries the hammering surface 1020, and the cap 1045 can have screw-fit, snap fit or the like. The cap also can have a living hinge to allow the hammering surface 1020 to be pivoted away from the fitting. In another embodiment shown in FIG. 22, the fitting 1040 can be recessed from the hammering surface 1020 within a notch or recess 1048 to allow hammering without contacting the recessed fitting 1040.

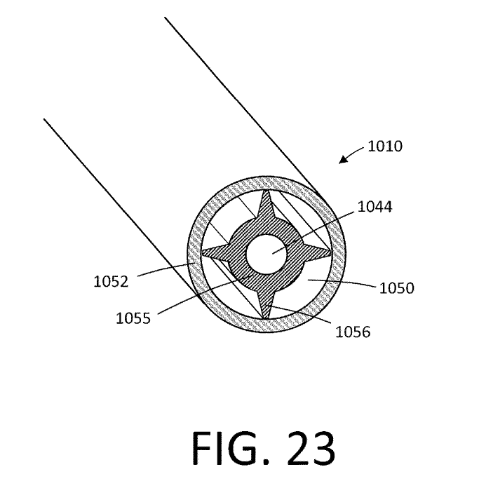

FIG. 23 is a sectional view of the extension portion 1010 of probe 1005 showing an insulative air space 1050 between outer sleeve 1052 and inner sleeve 1055 that carries the vapor delivery lumen 1044. The outer sleeve 1052 can be any metal tube suitable for driving into bone, such as a 10 ga. to 14 ga, stainless steel tube. The inner sleeve 1055 can be a high-temperature resistant biocompatible plastic such as PEEK with longitudinal or helical fins 1056 that support the sleeve 1055 centrally in the bore 1058 of outer sleeve 1052 to maintain the insulative air space. The air space 1050 can also comprise a sealed partial vacuum or can be configured with a pump mechanism to provide a partial vacuum at the time of use. The insulative space 1050 can also be provided by an aerogel filler instead of the fins 1056 on the inner sleeve 1055.

In a method of use, the probe shaft 1010 of FIGS. 19-20 is hammered into a vertebral body in a transpedicular approach as shown in FIG. 20. In another method, the shaft may be advanced parapedicularly. The physician can utilize bi-planar fluoroscopy to optimize the position of working end 1025. Thereafter, the physician can actuate the system to deliver vapor for a predetermined interval to ablate the basivertebral nerve, for example, 5 seconds or less; 10 seconds or less; 30 seconds or less; or 60 seconds or less. The working end can be configured to deliver vapor from vapor outlets that number from 1 to about 20 over a length of from 1 mm to 20 mm. The vapor can be expelled in pulses or in a continuous mode.

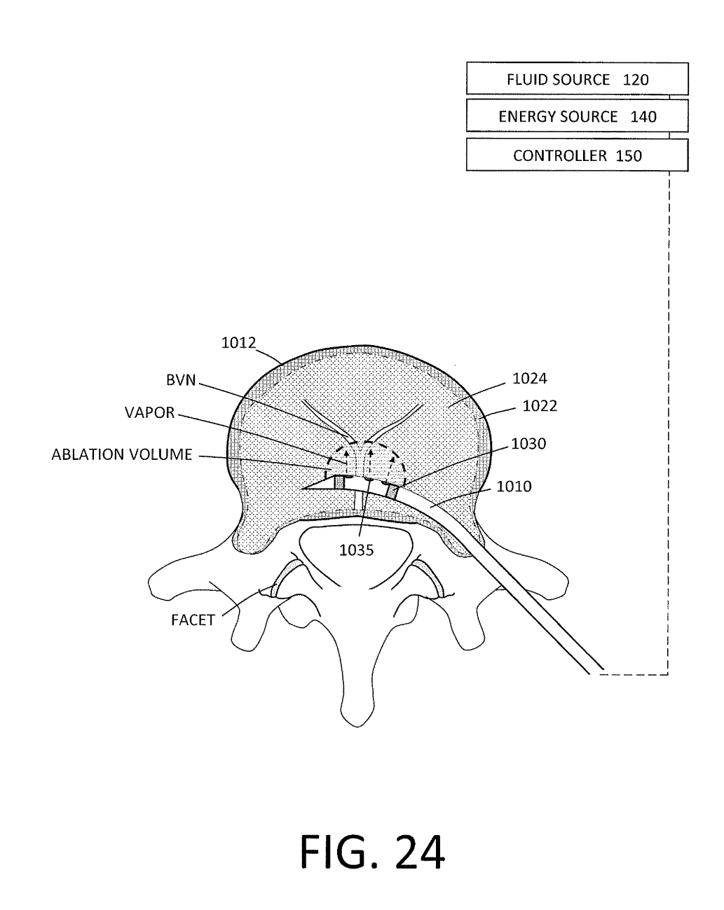

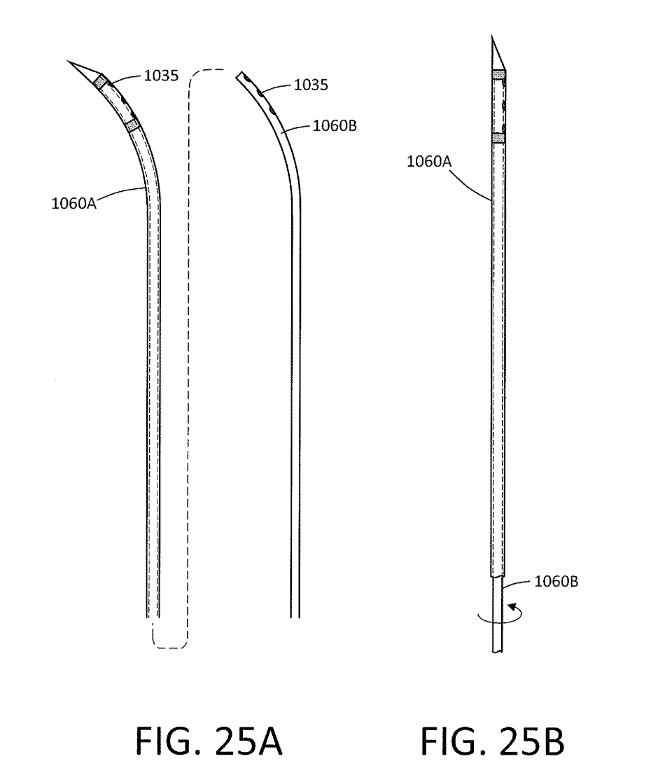

FIG. 24 illustrates another embodiment of system 1000 that is similar to that of FIGS. 19-20 except that the vapor delivery probe 1005′ has an extension member 1010 that carries a working end 1025 that is deflectable to allow its navigation to a posterior portion of the interior of a vertebral body. Such a deflectable working end can be configured with vapor outlets 1035 facing outwardly from the radius or curved axis of the working end to expel vapor anteriorly to ablate the basivertebral nerve BVN. Such an approach may be safer that expelling vapor in a posterior direction toward the spinal canal. This approach also may be safer in used in a method that does not include an aspiration component as described above. FIGS. 25A-25B show one system with a deflectable working end 1025 in which first and second sleeves, 1060A and 1060B, are fabricated of a shape memory alloy (e.g., Nitinol) and have cooperating curved memory shapes as indicated in FIG. 25A. The sleeves are designed so that 180° relative rotation of the sleeves can move the working end from the straight configuration of FIG. 25B in which both sleeves are stressed to a deflected position in which both sleeves are unstressed as in FIG. 25A. The inner sleeve 1060B can carry the vapor delivery lumen 1044, which can communicate with vapor outlets 1035 in both sleeves that align in a selected deflected configuration. The deflecting working end 1025 can be provided by other mechanisms known in the art, such as slotted tubes with pull-cables, concentric slotted tubes, hinged and segmented tubes and the like.

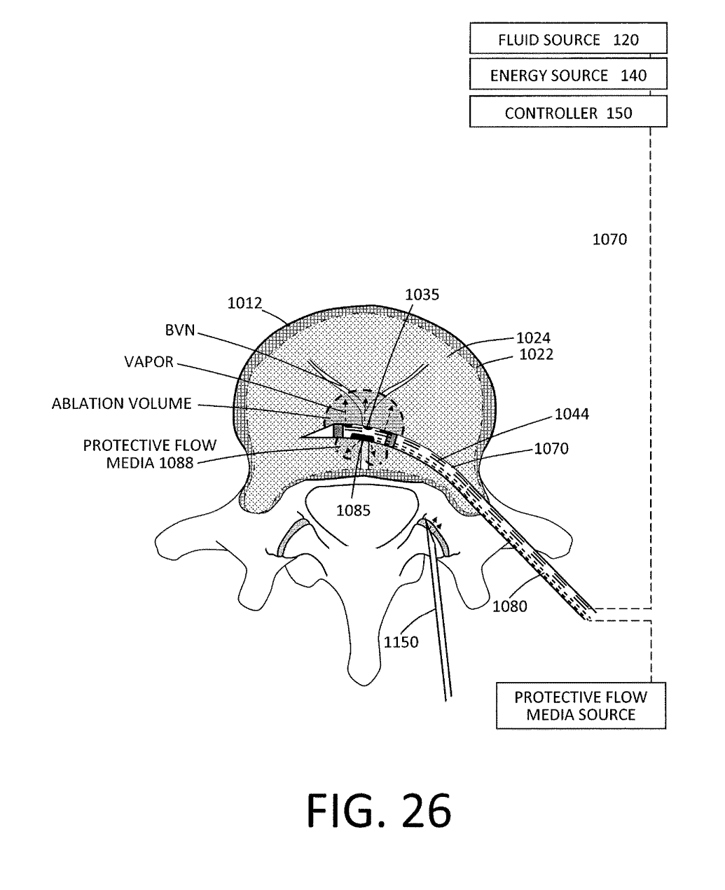

In another embodiment shown in FIG. 26, the probe extension member 1070 and working end 1025 can be configured for accessing any tissue in a straight or deflecting working end, and further can include vapor delivery lumen 1044 and outlets 1035 for delivering vapor as described previously. In addition, the extension portion carries a second lumen 1080 that communicates with second flow outlet(s) 1085 in the working end for expelling a second fluid from the working end. The at least one second outlet 1085 is spaced apart from the vapor outlets 1035 and is adapted for delivery of another functional fluid (gas, liquid, gel) to the region targeted for treatment, either prior to vapor delivery or contemporaneous with vapor delivery. In the method depicted in FIG. 26, the physician can inject a protective cooling fluid, cooling gas, or cryogenic gas to indicated at 1088 to protectively cool a region of tissue on one side of the working end before or concurrently with vapor delivery. In another embodiment, the second channel can be used for delivering a protective gel, or polymerizable liquid that turns into a gel or solid, to provide a convective barrier to vapor flow in the tissue. FIG. 26 shows the protective flow media 1088 on the opposite side of the vapor outlets. In another embodiment (not shown), the working end can carry vapor outlets 1035 on a shaft that are intermediate first and second sets of second outlets 1085 that are configured for injection of a protective flow media that serves as a convective barrier or cooling media.

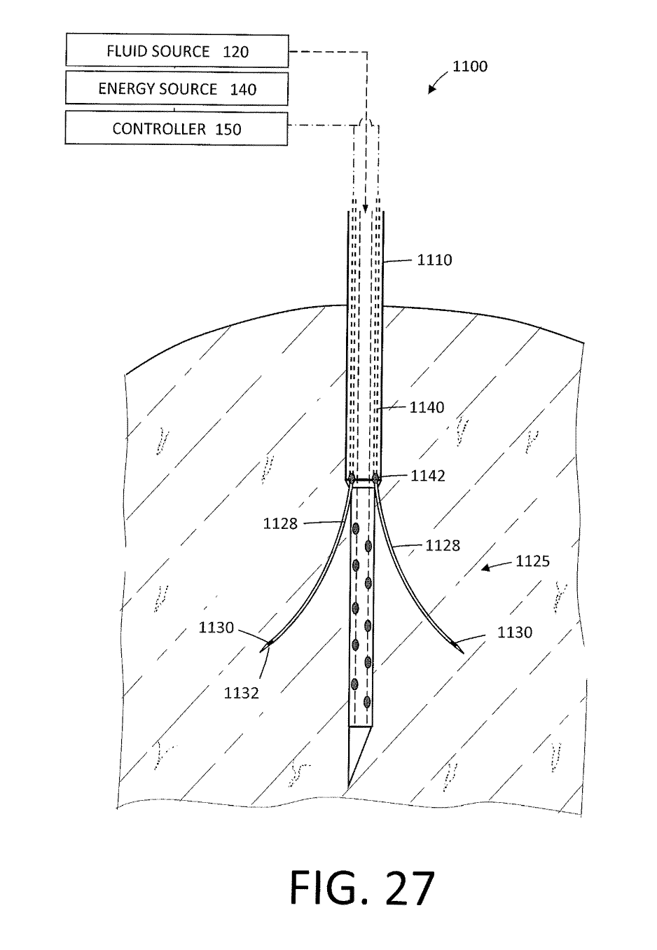

In another embodiment shown in FIG. 27, a system and probe 1100 with extension member 1110 and working end 1125 functions as described above with additional features comprising at least one temperature-sensing extendable member 1128 that extends from the working end into tissue a predetermined distance in soft tissue. In one embodiment, as shown in FIG. 27, there are two, three or four extendable members 1128 spaced around the shaft and configured for extension into soft tissue, with a thermocouple 1130 disposed on a distal end 1132 of each member. The thermocouples 1130 are operatively connected to the controller 150, and software can be provided to terminate or modulate vapor delivery when one or more thermocouples reach a peak temperature or a rate or acceleration of temperature increase. The extendable members further can have radiopaque marking for imaging to confirm or determine location. The objective would be to position the temperature sensors at the periphery of the tissue volume targeted for ablation. The extendable members can have a rectangular or flat cross-section or otherwise keyed to guide channels 1140 and apertures 1142 from which they extend to control the direction of deployment.

Referring to FIG. 27, a method of invention comprises the steps of (i) introducing an elongated probe into a patient body such that a working end with at least one vapor delivery outlet within or adjacent to the targeted tissue, (ii) deploying at least one extension member with temperature sensor in region proximate a periphery of the targeted tissue and monitoring temperature; (iii) delivering vapor through the working end and outlet(s) configured to apply ablative energy to the tissue, and (iv) controlling the applied energy in response to the monitored temperature. Thus, the temperature sensor, such as a thermocouple, has feedback circuitry coupled to the controller 150 for modulating, pulsing, or terminating energy delivery or vapor parameters. In another embodiment, the temperature sensors can be fiber optic probes configured for temperature measurement.

Referring back to FIG. 26, another method of invention comprises utilizing a vapor delivery tool or needle to access a zygapophyseal joint or paravertebral region and delivering vapor to ablate such tissue and nerves therein to treat spinal pain. Needle 1150 is shown schematically in FIG. 26 delivering vapor and ablative energy to the zygapophyseal joint.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed is:

1. A method of treating discogenic pain comprising:
applying energy to a flow media produce a water vapor media;
introducing the water vapor media into an intervertebral disc such that the vapor media releases energy into a treatment site in the intervertebral disc during a vapor to liquid transition to produce a therapeutic effect in the treatment site in the disc.

2. The method of claim 1 wherein the water vapor media has a temperature of at least 60 degrees C.

3. The method of claim 1 wherein the water vapor media has a mass average temperature of at least 60 degrees C.

4. The method of claim 1 wherein the treatment site includes tissue proximate at least one of a disc bulge, a disc protrusion, a disc extrusion and an annular tear or defect.

5. The method of claim 1 wherein the treatment site includes tissue containing sinuvertebral nerves.

6. The method of claim 1 wherein the treatment site includes a portion of the annulus fibrosus containing sinuvertebral nerves.

7. The method of claim 1 wherein the water vapor media includes a pharmacological agent.

8. The method of claim 1 wherein the water vapor media includes an analgesic.

9. The method of claim 1 wherein the effects include at least one of ablation of nerves, reduction of intradiscal pressure and shrinkage of disc tissue.

10. The method of claim 1 further comprising applying aspiration forces within the disc.

11. A method of treating pain comprising:
applying energy to a flow media to produce a condensable vapor media;
introducing the condensable vapor media proximate to an intraosseal nerve and where the energy ablates the intraosseal.

12. A method of treating spinal pain comprising introducing a vapor delivery tool into the interior of a vertebral body and delivering a vapor media wherein condensation of the vapor applies energy sufficient to ablate a basivertebral nerve.

13. The method of claim 12 wherein the vapor media is substantially water vapor.

14. A method of treating spinal pain comprising introducing a condensable vapor media proximate to a nerve in at least one of the interior of a disc, the interior of a vertebral body, a zygapophyseal joint and a paravertebral region wherein condensation of the vapor media applies energy to ablate said nerve.

15. The method of claim 14 wherein the nerve comprises sinuvertebral nerves.

16. The method of claim 15 wherein the nerve comprises a basivertebral nerve.

17. The method of claim 15 wherein the vapor media is substantially water vapor.

18. The method of claim 15 wherein the vapor media is at least 80% water vapor.

19. A method of ablating a targeted tissue volume in a patient body comprising:
introducing an elongated probe into a patient body that includes at least one vapor delivery outlet for delivering a condensable thermal vapor to the targeted tissue;
deploying at least one needle member with a temperature sensor into region proximate a periphery of the targeted tissue and monitoring temperature;
delivering the condensable vapor through the probe and at least one outlet to apply ablative energy to the tissue, and
controlling the applied energy in response to the monitored temperature.

21. The method of claim 20 wherein monitoring temperature is performed with a thermocouple temperature sensor.

22. The method of claim 20 wherein monitoring temperature is performed with a fiber optic temperature sensor system.

23. A method of ablating a targeted tissue volume in a patient body comprising:
providing an elongated vapor delivery tool having a working end with at least one vapor outlet for delivering a condensable vapor flow to the targeted tissue;
delivering a vapor flow through the at least one outlet into the targeted tissue; and
allowing a flexible member disposed around a proximal tool portion to expand when contacted by vapor flow to thereby prevent unwanted retrograde vapor flow.

24. A system for ablating a tissue within a vertebral body or intervertebral disc, the system comprising:
an introducer having a blunt tip; and
a vapor delivery needle coupled to a fluid media source, where the vapor delivery needle is configured to apply a vaporization energy to the fluid media and where the vaporization energy exceeds a heat of vaporization of the fluid media therein to convert the fluid media to a vapor media, where the vapor delivery needle comprises at least one vapor delivery port to direct the vapor media to the tissue such that when the vapor media contacts the tissue energy transfer occurs from the vapor media to the tissue, the vapor delivery needle having a sharp tip sufficient to advance through the a vertebral body or intervertebral disc.

25. The system of claim 24 further comprising a controller coupled to the vapor delivery needle, where the controller is configured to controlling delivery of the vaporization energy to maintain a treatment temperature of the tissue above an ablation temperature of the tissue and below a transformation temperature of the tissue, such that the energy ablates the tissue allowing the tissue to subsequently be resorbed by the body.

26. The system of claim 24, where the needle comprises a shape memory alloy material.

26. The system of claim 24, where the needle comprises a shape memory alloy material.

27. The system of claim 24, where a distal portion of the vapor delivery needle is curved.

28. The system of claim 27, where the at least one vapor delivery ports is located on an interior radius of the curved portion.

29. The system of claim 27, where the at least one vapor delivery port comprise a plurality of vapor delivery ports that are configured to emit vapor in a radial arc.

30. The system of claim 24, further comprising a negative pressure source coupled to a sleeve extending through the introducer and the vapor delivery needle is located within the sleeve.

31. The system of claim 30, where the sleeve further a lumen having dual-lead helical elements having an edge that contacts the vapor delivery needle, such that elements form at least one helical channel that winds around the vapor delivery needle.

32. The system of claim 31, further comprising a source of cooling fluid fluidly coupled to the helical channel.

33. The system of claim 30, further comprising an elastomeric sleeve having a proximal portion affixed to the vapor delivery needle, where a distal portion of the elastomeric sleeve is able to expand away from the vapor delivery needle.

34. The system of claim 24, further comprising a proximal handle having a hammering surface configured to allow application of an impact force theron to drive the sharp tip of the vapor delivery needle tipped through hard tissue.

34. The system of claim 24, further comprising at least one radiopaque marking on the vapor delivery needle and proximate to the vapor delivery outlets.

34. The system of claim 24, further comprising an extension member located within the vapor delivery needle, where the vapor delivery needle member comprises a deflectable working end.

35. The system of claim 34, where the deflectable working end comprises a pre-curved shape and where the extension member comprises a pre-curved shape, and where the extension member and vapor delivery needle are rotatable relative to each other such that rotation of the vapor delivery needle relative to the extension member deflects the deflectable working end of the vapor delivery needle.

36. The system of claim 24, further comprising at least one temperature sensing member extendable from the introducer.

37. The system of claim 36, where the at least one temperature sensing member comprises a plurality of temperature sensing members.

38. The system of claim 36, where the at least one temperature sensing is operatively connected to a controller to provide temperature feedback to the controller to terminate or modulate vapor delivery upon reaching a peak temperature or a rate or acceleration of temperature increase.
